(19) United States
(12) Patent Application Publication
(10) Pub. No.: US 2003/0018185 A1
(43) Pub. Date: Jan. 23, 2003

(54) Plant microsatellite markers and methods for their use

(76) Inventors: Ilkka Havukkala, Auckland (NZ); Leonard Bloksberg, Auckland (NZ); Matthew Glenn, Auckland (NZ)

Correspondence Address: SPECKMAN LAW GROUP, 1501 WESTERN AVE, SUITE 100, SEATTLE, WA, 98101, US

(21) Appl. No.: 10/062,727

(22) Filed: Feb. 5, 2002

Related U.S. Application Data
(63) Continuation-in-part of application No. 09/105,307, filed on Jun. 25, 1998.

(51) Int. Cl. C12Q 1/68; C07H 21/04

(52) U.S. Cl. 536/23.6; 435/6

(57) ABSTRACT

Microsatellite sequences and associated flanking sequences isolated from pine and eucalyptus are provided, together with methods for the use of such sequences in the detection of polymorphic genetic markers. Kits comprising oligonucleotide primers and/or hybridization probes for use in such methods are also provided.

### REFERENCE TO RELATED APPLICATIONS

This is application is a continuation-in-part of U.S. patent application Ser. No. 09/105,307, filed Jun. 25, 1998.

### REFERENCE TO SEQUENCE LISTING SUBMITTED ON COMPACT DISC

This application incorporates by reference in its entirety the Sequence Listing that is provided in duplicate on compact discs that accompany the application. Each CD contains the following file: 1006CIP, having a date of creation of Feb. 4, 2002 and a file size of 1.42 MB.

### TECHNICAL FIELD OF THE INVENTION

The present invention relates to the field of DNA markers useful in genetic analysis. More specifically, the present invention relates to plant microsatellite markers, and methods for using such markers in the identification of polymorphisms and in genome mapping.

### BACKGROUND OF THE INVENTION

Microsatellites are lengths of DNA found in mostly non-coding areas of genomes of various organisms. They are composed of a number of tandemly repeated short nucleotide motifs, or repeat units. Microsatellites (also referred to as simple sequences, simple sequence repeats (SSRs), simple repetitive DNA sequences, short tandem repeats (STRs) or simple sequence motifs (SSMs)) have been isolated from many eukaryotic species, and are ubiquitous in plants (Wang Z. J., Weber L., Zhong G. & Tanksley S. D. 1994 Theor. Appl. Genet. 88:1-6), with specific microsatellite sequences being interspersed at many locations within the genome. The repeat nucleotide motifs found within microsatellites are generally 1-5 basepairs long, but can be longer. The number of repeat units found in a specific microsatellite varies from approximately 5 to 50, with each microsatellite being flanked with non-repetitive nucleotide sequence. The precise number of repeat units found within a microsatellite can vary among species and even among closely related individuals. Thus different alleles of the same gene may share the same flanking sequences, but contain a different number of repeat units in the middle. Sometimes the repeat is slightly imperfect, but with a still recognizable length of tandem repeat area and type of repeat unit. These DNA variations, or polymorphisms, may be usefully employed as markers for the identification of an individual's DNA and for genome mapping, with the uniqueness of the flanking sequences assisting in making the markers more informative and more specific.

Two classes of markers commonly used in genome mapping programs are restriction fragment length polymorphisms (RFLP) and random amplified polymorphic DNAs (RAPD). In comparison with microsatellites, however, the use of RFLP requires tedious restriction enzyme digestions of large amounts of DNA and separation of many digests from different individuals in parallel using gel electrophoresis. RAPDs are faster and cheaper to develop than microsatellites, but are often less informative, in part because they are generally not considered to be applicable in other cultivars or species, and they often show less polymorphism.

In humans, microsatellite polymorphisms have been used widely for individual identification in, for example, paternity and forensic cases, and for mapping of genes correlating with genetic diseases. For example, U.S. Pat. No. 5,364,759 discloses typing assays for fingerprinting of human individuals for forensic and medical purposes, as well as techniques for identifying microsatellite sequences from DNA databases. Specific trimeric and tetrameric short tandem repeats (STRs) present in the human genome with characteristics suitable for inclusion in DNA profiling assays are also disclosed. U.S. Pat. No. 5,582,979 provides a large variety of specific sequences, isolated from human genomic DNA, which flank CA and GT dinucleotide repeats for use in forensic and paternity tests employing polymorphisms in the repeat area.

U.S. Pat. No. 5,580,728 discloses a method and automated system for genotyping using amplified DNA sequences containing repetitive sequences showing polymorphism between DNA samples. This patent describes techniques for automated data acquisition and interpretation using short tandem repeats (STRs) and the steps required to build genetic maps based on such polymerase chain reaction (PCR)-amplified markers. U.S. Pat. No. 5,573,912 describes a protocol for obtaining novel short tandem repeat regions from DNA using size-separated restriction enzyme digests, followed by hybridization with genomic DNA of the same species, and comparison of the hybridization pattern with that obtained using known probes containing variable tandem repeat regions. No specific sequences of immediate utility for genotyping are disclosed.

U.S. Pat. Nos. 5,369,004 and 5,378,602 disclose specific sequences suitable as PCR primers for DNA repeat polymorphism detection in humans for medical purposes and genetic mapping. U.S. Pat. No. 5,650,277 discloses a method of determining the exact number of oligonucleotide repeats within a microsatellite, wherein each repeat is two or three nucleotides long. This patent does not teach any specific primers, but requires previous determination of the repeat sequence within the microsatellite or of sequences flanking the microsatellite.

None of the microsatellite sequences and associated flanking sequences identified in humans or other mammals are likely to be useful for detecting plant DNA polymorphisms, since the abundance and types of various kinds of DNA repeat motifs varies between plants and animals.

Microsatellites have been used for genome mapping of various plants, including rice, maize, soybean, barley and tomato, and are therefore becoming important tools for use in the preparation of genome maps. For a review of the use of DNA repeat motifs in plant genome mapping see Zhao et al. (Applications of repetitive DNA sequences in plant genome analysis.—pp. 111-125, Chapter 10 in: Paterson, AH (ed.), Genome Mapping in Plants. R. G. Landes Co., New York, 1996). In addition to genetic mapping, microsatellites may be employed in physical mapping. For example, some types of repeats may show a specific distribution on the chromosomes (Schmidt T & Heslop-Harrison J S, 1996 Proc. Natl. Acad. Sci. USA 93(16): 8761-8765), so that different microsatellites may be useful in physical mapping of different areas of the genome.

Microsatellites have also been used for fingerprinting of many agricultural plants, as well as evaluating genetic diversity between plant cultivars, subspecies and so on. The main advantage of microsatellites is that they are often highly polymorphic, even within a species and cultivar. In addition, the microsatellite flanking sequences are often locus-specific thus providing a specific probe for reliably isolating that genome region. Examples of the use of microsatellites in plant identification include grapevine cultivar identification and evaluation of the genetic relatedness of cultivars (Thomas M R, Cain P, Scott N S, 1994 Plant Mol. Biol. 25(6): 939-949); identifying individuals of wild yam for common parents in natural populations (Terauchi R & Konuma A, 1994 Genome 37(5): 794-801); variety identification of leaf mustard gernplasm (Bhatia S, Das S, Jain A, Lakshmikumaran M., 1995 Electrophoresis 16(9): 1750-1754); identification of chickpea varieties (Sharma P C, Huttel B, Winter P, Kahl G, Gardner R C, Weising K., 1995 Electrophoresis 16(9):1755-1761); maize cultivar germplasm genetic analysis (Taramino G & Tingey S., 1996 Genome 39(2): 277-287); and evaluation of within-cultivar variation of genetic diversity in rice (Olufowote J O, Xu Y, Chen X, Park W D, Beachell H M, Dilday R H, Goto M, McCouch S R., 1997 Genome 40(3): 370-380).

Microsatellite markers are being increasingly employed to locate specific, economically useful genes in plant genomes by linkage analysis. For example, STRs were used to map a microsatellite marker close to the rice Rf1 gene, a fertility restorer gene essential for hybrid rice production, by PCR amplification and linkage analysis of microsatellite polymorphism (Akagi H, Yokozeki Y, Inagaki A, Nakamura A, Fujimura T., 1996 Genome 39(6): 1205-1209). This marker will be employed not only in breeding fertility restorer and maintainer lines, but also in managing the purity of hybrid rice seeds.

The use of short tandem repeat DNA sequences in tree genetics is just beginning, with microsatellite markers recently being developed for oak (Dow B D, Ashely M B & Howe H F., 1995 Theor. Appl. Genet. 91: 137-141), Citrus (Kijas J M H, Fowler J C S & Thomas M R., 1995 Genome 38: 349-355), Pinus radiata (Smith D N & Devey M E, 1994 Genome 37: 977-983), Pinus sylvestris, Pinus strobus (Echt C S, May-Marquardt P, Hseih M & Zahorchak R., 1996 Genome 39: 1102-1108), Pinus elliottii (Doudrick R L, 1996 Symp. Soc. Exp. Biol. 50: 53-60), and Pinus taeda (Echt C S & May-Marquardt P, 1997 Genome 40: 9-17). The need for the isolation of DNA sequences flanking microsatellites is rapidly increasing with the start of tree genome mapping projects around the world. These markers will be especially valuable for the Pinus species, which have large genomes making isolation of RAPD or RFLP probes more difficult (Neale, D B & Sederoff, R R, 1996, pp. 309-319 in Chapter 22 in: Paterson, AH (ed.), Genome Mapping in Plants, R. G. Landes Co., New York, 1996).

Conventional techniques for the development of microsatellite markers are expensive and time-consuming, and generally require the following steps:

a) isolation of repeat-containing DNA clones by screening genomic DNA or cDNA libraries with repetitive DNA probes, and detecting polymorphic bands from electrophoresis gels;

b) isolation and sequencing of the repeat-containing DNA fragments;

c) designing specific PCR primers flanking the repeat for specific amplification of the specific repeat; and

d) scoring for polymorphism in the amplification products (typically, varying size DNA fragments in an agarose gel).

A limited number of microsatellite markers are available commercially, for example from Research Genetics Inc. (Huntsville, Ala., USA).

The time, effort and great expense needed to identify and isolate microsatellite sequences is a serious limitation for the expanded use of microsatellites in plant genetics. This is particularly true for plant species with very large genomes, such as wheat and pine. Protocols for the preparation of plant DNA libraries enriched for microsatellite sequences have recently been developed (Edwards K J, Barker J H A, Daly A, Jones C & Karp A, 1996 BioTechniques 20(5): 758-760), but the lack of significant numbers of microsatellite markers is still limiting progress in plant genetic mapping and DNA fingerprinting. There thus remains a need in the art for plant microsatellite markers for use in plant genome mapping and breeding programs.

### SUMMARY OF THE INVENTION

Briefly, the present invention provides isolated microsatellite sequences obtainable from pine and eucalyptus, together with flanking sequences specific to the inventive microsatellite sequences. Methods for the use of probes and primers designed from such microsatellite and flanking sequences, together with kits comprising such probes and primers are also provided.

In a first aspect, the present invention provides isolated polynucleotides comprising at least one microsatellite repeat and at least one associated flanking sequence. In one embodiment, the isolated polynucleotides of the present invention comprise a sequence selected from the group consisting of: (a) sequences provided in SEQ ID NO: 1-1054; (b) sequences complementary to sequences provided in SEQ ID NO: 1-1054; and (c) variants of a sequence of (a) or (b) as defined below. In a further embodiment, the present invention provides isolated polynucleotides comprising a sequence selected from the group consisting of: (a) left flanking sequences of a sequence provided in SEQ ID NO: 1-1054; (b) right flanking sequences of a sequence provided in SEQ ID NO: 1-1054; (c) sequences complementary to a sequence of (a) or (b); and (d) variants of a sequence of (a), (b) or (c), as defined below. The left and right flanking sequences for each of the inventive sequences are identified by residue number in Table 1 below.

In a further aspect, the invention provides novel microsatellites, comprising a sequence selected from the group consisting of: (a) at least three contiguous repeats of a sequence provided in SEQ ID NO: 1055; (b) at least three contiguous repeats of a sequence provided in SEQ ID NO: 1056; (c) at least three contiguous residues of a sequence provided in SEQ ID NO: 1057; and (d) variants of a sequence of (a), (b) or (c).

The inventive polynucleotide sequences may be used to design oligonucleotides for use as probes for the detection and isolation of microsatellite-containing DNA by hybridization and as primers for amplification of microsatellite-containing DNA by PCR. In specific embodiments, the oligonucleotide probes and/or primers comprise at least about 6 contiguous residues, more preferably at least about 10 contiguous residues and most preferably at least about 20 contiguous residues of a polynucleotide sequence of the present invention.

In other aspects, methods for the detection of polymorphic genetic markers in a subject are provided, together with kits for use in such methods. Generally, the inventive methods comprise isolating genomic or other DNA (for example, cDNA) from a sample and assaying for the presence of a polymorphic genetic marker using at least one oligonucleotide probe or primer of the present invention. The isolated DNA may be analyzed by means of a hybridization assay, in which the DNA is contacted with the polynucleotide probe under standard hybridization conditions. DNA molecules that hybridize with the polynucleotide probe are isolated, separated according to size using, for example, gel electrophoresis, and analyzed for the presence of a polymorphic genetic marker.

In a preferred embodiment, the isolated genomic DNA is subjected to polymerase chain reaction using a primer pair comprising at least one inventive oligonucleotide primer, to provide amplified DNA molecules. The amplified DNA molecules are subsequently separated according to size, such as by gel electrophoresis, and the presence or absence of the polymorphic genetic marker and degree of polymorphism is determined by comparing various samples from, for example, different tissues, individuals or populations. Other types of assays employing probes of repeat flanking sequences on solid-base supports, such as charged nylon membranes, sephadex beads or DNA chips, and subsequent detection of the length of the adjoining repeat are also contemplated by the present invention.

In general, the polymorphic genetic markers detected using the inventive methods represent variations in the number and exact sequence of repeat units found within a microsatellite. Preferably, the DNA is isolated from a plant or from the fruit or seeds thereof. In one embodiment, the subject being examined for the presence and degree of polymorphism is a woody plant, most preferably selected from the group consisting of the genus Eucalyptus and Pinus.

The inventive microsatellite-containing sequences may thus be usefully employed for variety identification and protection, monitoring of seed purity and origin, genome mapping and physical mapping of genomes, and positional cloning of economically important genes located near the polymorphic markers. In addition, the inventive sequences may be used in transforming various organisms for either influencing a heritable trait or marking them by heterologous identity markers.

The present invention also provides a computer readable medium on which is stored at least one polynucleotide sequence, or oligonucleotide probe or primer sequence, of the present invention. Suitable computer readable media include floppy disks, hard drives, CD-ROM disks and magnetic tape. The sequences may be stored using any computer program known to those of skill in the art.

The above-mentioned and additional features of the present invention and the manner of obtaining them will become apparent, and the invention will be best understood by reference to the following more detailed description.

### DETAILED DESCRIPTION OF THE INVENTION

The present invention provides isolated microsatellite DNA sequences and DNA sequences flanking such microsatellites, such sequences being obtainable from eucalyptus and pine species. Specifically, the present invention provides isolated polynucleotides comprising a nucleotide sequence of SEQ ID NO: 1-1054, a complement of a sequence of SEQ ID NO: 1-1054, or a variant thereof. Each of the sequences provided in SEQ ID NO: 1-1054 is composed of a number of tandemly repeated motifs of between 1 and 10 nucleotides located next to non-repetitive flanking sequence(s) of up to a few hundred nucleotides in length. Table 1 identifies the left, or 3′, flanking sequence; repeat region; and right, or 5′, flanking sequence for each of SEQ ID NO: 1-1054 by residue number.

TABLE 1

| | left flanking | | right flanking |
|---|---|---|---|
| SEQ ID NO: | sequence | repeat motif | sequence |
| 1 | 1-43 | 44-57 | 58-80 |
| 2 | 1-74 | 75-98 | 99-130 |
| 3 | 1-68 | 69-89 | 90-116 |
| 4 | 1-23 | 24-50 | 51-79 |
| 5 | 1-83 | 84-107 | 108-171 |
| 6 | 1-111 | 112-135 | 136-210 |
| 7 | 1-144 | 145-164 | 165-239 |
| 8 | 1-56 | 57-77 | 78-121 |
| 9 | 1-14 | 15-38 | 39-117 |
| 10 | 1-59 | 60-80 | 81-265 |
| 11 | 1-14 | 15-28 | 29-114 |
| 12 | 1-104 | 105-136 | 137-151 |
| 13 | 1-16 | 17-30 | 31-111 |
| 14 | 1-22 | 23-66 | 67-300 |
| 15 | 1-81 | 82-95 | 96-114 |
| 16 | 1-73 | 74-89 | 90-128 |
| 17 | 1-94 | 95-112 | 113-142 |
| 18 | 1-12 | 13-24 | 25-409 |
| 19 | 1-16 | 17-30 | 31-171 |
| 20 | 1-139 | 140-163 | 164-274 |
| 21 | 1-23 | 24-53 | 54-126 |
| 22 | 1-127 | 128-149 | 150-237 |
| 23 | 1-65 | 66-77 | 78-136 |
| 24 | 1-81 | 82-105 | 106-122 |
| 25 | 1-72 | 73-92 | 93-129 |
| 26 | 1-196 | 197-220 | 221-267 |
| 27 | 1-13 | 14-39 | 40-180 |
| 28 | 1-125 | 126-133 | 0 |
| 29 | 1-59 | 60-71 | 72-268 |
| 30 | 1-49 | 50-65 | 66-136 |
| 31 | 1-259 | 260-277 | 278-469 |
| 32 | 1-11 | 12-50 | 51-454 |
| 33 | 0 | 1-10 | 11-196 |
| 34 | 1-113 | 114-131 | 132-283 |
| 35 | 1-96 | 97-120 | 121-264 |
| 36 | 1-82 | 83-106 | 107-263 |
| 37 | 1-11 | 12-47 | 48-193 |
| 38 | 0 | 1-12 | 13-348 |
| 40 | 1-16 | 17-34 | 35-251 |
| 41 | 0 | 1-12 | 13-179 |
| 42 | 1-81 | 82-102 | 103-146 |
| 43 | 1-264 | 265-294 | 0 |
| 44 | 1-164 | 165-182 | 183-329 |
| 45 | 1-45 | 46-57 | 58-421 |
| 46 | 1-21 | 22-39 | 40-261 |
| 47 | 0 | 1-14 | 15-262 |
| 48 | 1-34 | 35-66 | 67-329 |
| 49 | 1-35 | 36-56 | 57-232 |
| 50 | 1-97 | 98-109 | 110-219 |
| 51 | 0 | 1-24 | 25-352 |
| 52 | 1-31 | 32-45 | 46-237 |
| 53 | 1-70 | 71-91 | 92-239 |
| 54 | 0 | 1-12 | 13-317 |
| 55 | 1-288 | 289-306 | 307-458 |
| 56 | 1-44 | 45-80 | 81-424 |
| 57 | 1-14 | 15-34 | 35-346 |
| 58 | 1-15 | 16-35 | 36-328 |
| 59 | 1-38 | 39-52 | 53-188 |
| 60 | 1-14 | 15-34 | 35-236 |
| 61 | 1-62 | 63-77 | 78-227 |
| 62 | 0 | 1-10 | 11-233 |
| 63 | 1-67 | 68-79 | 80-296 |
| 64 | 0 | 1-16 | 17-342 |
| 65 | 1-31 | 32-69 | 70-396 |
| 66 | 1-71 | 72-92 | 93-271 |
| 67 | 1-18 | 19-32 | 33-373 |
| 68 | 1-25 | 26-49 | 50-335 |
| 69 | 1-80 | 81-92 | 93-288 |
| 70 | 1-71 | 72-95 | 96-357 |
| 71 | 0 | 1-12 | 13-316 |
| 72 | 0 | 1-20 | 21-295 |
| 73 | 1-145 | 146-165 | 166-318 |
| 74 | 0 | 1-10 | 11-310 |
| 75 | 1-14 | 15-38 | 39-338 |
| 76 | 0 | 1-18 | 19-434 |
| 77 | 0 | 1-12 | 13-510 |
| 78 | 1-47 | 48-77 | 78-402 |
| 79 | 1-10 | 11-20 | 21-353 |
| 80 | 1-185 | 186-200 | 201-312 |
| 81 | 0 | 1-12 | 13-294 |
| 82 | 1-11 | 12-19 | 20-232 |
| 83 | 1-29 | 30-50 | 51-321 |
| 84 | 0 | 1-20 | 21-268 |
| 85 | 1-11 | 12-31 | 32-330 |
| 86 | 1-14 | 15-34 | 35-281 |
| 87 | 1-26 | 27-62 | 63-425 |
| 88 | 1-312 | 313-330 | 331-454 |
| 89 | 1-89 | 90-107 | 108-315 |
| 90 | 1-185 | 186-200 | 201-310 |
| 91 | 0 | 1-10 | 11-402 |
| 92 | 1-34 | 35-46 | 47-346 |
| 93 | 1-80 | 81-104 | 105-287 |
| 94 | 1-218 | 219-252 | 0 |
| 95 | 0 | 1-14 | 15-251 |
| 96 | 1-247 | 248-265 | 266-322 |
| 97 | 1-74 | 75-96 | 97-419 |
| 98 | 1-57 | 58-78 | 79-291 |
| 99 | 1-20 | 21-46 | 47-352 |
| 100 | 0 | 1-24 | 25-275 |
| 101 | 0 | 1-18 | 19-284 |
| 102 | 0 | 1-12 | 13-344 |
| 103 | 1-169 | 170-201 | 202-378 |
| 104 | 1-20 | 21-50 | 51-334 |
| 105 | 1-30 | 31-48 | 49-379 |
| 106 | 1-144 | 145-164 | 165-338 |
| 107 | 1-329 | 330-347 | 348-379 |
| 108 | 1-224 | 225-236 | 237-291 |
| 109 | 1-165 | 166-179 | 180-190 |
| 110 | 1-26 | 27-56 | 57-284 |
| 111 | 1-44 | 45-62 | 63-444 |
| 112 | 1-61 | 62-83 | 84-400 |
| 113 | 1-48 | 49-60 | 61-245 |
| 115 | 1-12 | 13-26 | 27-283 |
| 116 | 0 | 1-10 | 11-258 |
| 117 | 1-178 | 179-202 | 203-440 |
| 118 | 1-311 | 312-329 | 330-424 |
| 119 | 0 | 1-42 | 43-125 |
| 120 | 1-44 | 45-80 | 81-343 |
| 121 | 1-80 | 81-92 | 93-383 |
| 122 | 1-34 | 35-52 | 53-392 |
| 123 | 0 | 1-10 | 11-255 |
| 124 | 1-35 | 36-56 | 57-222 |
| 125 | 0 | 1-16 | 17-256 |
| 126 | 1-28 | 29-48 | 49-403 |
| 127 | 1-46 | 47-58 | 59-295 |
| 128 | 0 | 1-8 | 9-120 |
| 129 | 0 | 1-24 | 25-347 |
| 130 | 1-40 | 41-58 | 59-283 |
| 131 | 1-14 | 15-34 | 35-198 |
| 132 | 1-73 | 74-85 | 86-172 |
| 133 | 1-21 | 22-39 | 40-262 |
| 134 | 1-276 | 277-294 | 295-367 |
| 135 | 1-166 | 167-184 | 185-322 |
| 136 | 1-30 | 31-63 | 64-360 |
| 137 | 1-45 | 46-57 | 58-294 |
| 138 | 0 | 1-24 | 25-330 |
| 139 | 1-12 | 13-26 | 27-406 |
| 140 | 0 | 1-12 | 13-365 |
| 141 | 1-307 | 308-327 | 328-375 |
| 142 | 0 | 1-10 | 11-277 |
| 143 | 1-83 | 84-107 | 108-318 |
| 144 | 1-48 | 49-57 | 58-193 |
| 145 | 1-164 | 165-182 | 183-227 |
| 146 | 1-44 | 45-80 | 81-221 |
| 147 | 0 | 1-20 | 21-343 |
| 148 | 1-51 | 52-67 | 68-328 |
| 149 | 1-71 | 72-95 | 96-316 |
| 150 | 1-14 | 15-34 | 35-197 |
| 151 | 0 | 1-24 | 25-319 |
| 152 | 1-12 | 13-30 | 31-329 |
| 153 | 1-41 | 42-59 | 60-182 |
| 154 | 1-16 | 17-34 | 35-211 |
| 155 | 0 | 1-12 | 13-365 |
| 156 | 1-46 | 47-76 | 77-356 |
| 157 | 1-52 | 53-73 | 74-266 |
| 158 | 1-20 | 21-34 | 35-173 |
| 160 | 1-166 | 167-184 | 185-262 |
| 161 | 1-12 | 13-32 | 33-147 |
| 162 | 1-286 | 287-304 | 305-390 |
| 163 | 1-12 | 13-26 | 27-349 |
| 164 | 1-164 | 165-182 | 183-328 |
| 165 | 1-20 | 21-40 | 41-311 |
| 166 | 1-18 | 19-34 | 35-337 |
| 167 | 1-44 | 45-65 | 66-277 |
| 168 | 0 | 1-14 | 15-243 |
| 169 | 1-145 | 146-165 | 166-302 |
| 170 | 1-167 | 168-177 | 178-203 |
| 171 | 1-89 | 90-101 | 102-216 |
| 172 | 1-64 | 65-82 | 83-291 |
| 173 | 0 | 1-12 | 13-152 |
| 174 | 0 | 1-18 | 19-387 |
| 175 | 1-185 | 186-203 | 204-229 |
| 176 | 1-34 | 35-67 | 68-274 |
| 177 | 1-158 | 159-176 | 177-254 |
| 178 | 0 | 1-12 | 13-298 |
| 179 | 1-27 | 28-51 | 52-333 |
| 180 | 1-167 | 168-176 | 177-196 |
| 181 | 0 | 1-16 | 17-353 |
| 182 | 1-34 | 35-52 | 53-314 |
| 183 | 1-23 | 24-39 | 40-328 |
| 184 | 1-111 | 112-135 | 136-367 |
| 185 | 1-44 | 45-74 | 75-367 |
| 186 | 1-16 | 17-32 | 33-285 |
| 187 | 1-161 | 162-175 | 176-311 |
| 188 | 1-68 | 69-90 | 91-398 |
| 189 | 1-30 | 31-60 | 61-239 |
| 190 | 0 | 1-18 | 19-349 |
| 191 | 1-26 | 27-42 | 43-276 |
| 192 | 1-105 | 106-114 | 115-325 |
| 193 | 1-29 | 30-50 | 51-280 |
| 194 | 1-19 | 20-35 | 36-190 |
| 195 | 1-256 | 257-288 | 0 |
| 196 | 1-10 | 11-44 | 45-398 |
| 197 | 1-45 | 46-57 | 58-366 |
| 198 | 1-58 | 59-82 | 83-347 |
| 199 | 1-33 | 34-45 | 46-364 |
| 200 | 1-42 | 43-54 | 55-407 |
| 201 | 1-100 | 101-124 | 125-400 |
| 202 | 1-19 | 20-33 | 34-170 |
| 203 | 1-42 | 43-60 | 61-279 |
| 204 | 1-19 | 20-39 | 40-318 |
| 205 | 1-54 | 55-66 | 67-234 |
| 206 | 1-137 | 138-159 | 160-459 |
| 207 | 0 | 1-100 | |
| 208 | 1-58 | 59-74 | 75-380 |
| 209 | 1-21 | 22-42 | 43-468 |
| 210 | 1-177 | 178-198 | 199-429 |
| 211 | 1-18 | 19-36 | 37-450 |
| 212 | 1-40 | 41-70 | 71-469 |
| 213 | 0 | 1-25 | 26-482 |
| 214 | 1-21 | 22-39 | 40-341 |
| 215 | 1-158 | 159-176 | 177-232 |
| 216 | 1-39 | 40-57 | 58-339 |
| 217 | 1-26 | 27-38 | 39-478 |
| 218 | 0 | 1-16 | 17-477 |
| 219 | 1-78 | 79-105 | 106-231 |
| 220 | 0 | 1-18 | 19-409 |
| 221 | 0 | 1-16 | 17-447 |
| 222 | 1-88 | 89-109 | 110-274 |
| 223 | 0 | 1-14 | 15-143 |
| 224 | 1-242 | 243-269 | 270-450 |
| 225 | 1-190 | 191-202 | 203-367 |
| 226 | 1-240 | 241-276 | 277-483 |
| 227 | 0 | 1-32 | 33-433 |
| 228 | 1-51 | 52-69 | 70-331 |
| 229 | 1-80 | 81-96 | 97-390 |
| 230 | 1-16 | 17-34 | 35-457 |
| 231 | 1-18 | 19-34 | 35-356 |
| 232 | 1-44 | 45-62 | 63-321 |
| 233 | 1-345 | 346-367 | 368-410 |
| 234 | 1-51 | 52-69 | 70-500 |
| 235 | 1-31 | 32-59 | 60-492 |
| 236 | 1-17 | 18-41 | 42-525 |
| 237 | 1-15 | 16-33 | 34-383 |
| 238 | 1-40 | 41-70 | 71-483 |
| 239 | 1-52 | 53-66 | 67-221 |
| 240 | 1-40 | 41-58 | 59-426 |
| 241 | 1-105 | 106-126 | 127-405 |
| 242 | 1-109 | 110-127 | 128-485 |
| 243 | 1-39 | 40-55 | 56-483 |
| 244 | 1-176 | 177-197 | 198-360 |
| 245 | 0 | 1-12 | 13-432 |
| 246 | 0 | 1-16 | 17-489 |
| 247 | 0 | 1-32 | 33-438 |
| 248 | 1-88 | 89-109 | 110-468 |
| 249 | 1-18 | 19-36 | 37-381 |
| 250 | 1-75 | 76-101 | 102-512 |
| 251 | 1-77 | 78-104 | 105-410 |
| 252 | 1-18 | 19-36 | 37-470 |
| 253 | 1-18 | 19-36 | 37-479 |
| 254 | 1-23 | 24-57 | 58-170 |
| 255 | 1-15 | 16-33 | 34-351 |
| 256 | 0 | 1-16 | 17-413 |
| 257 | 0 | 1-16 | 17-465 |
| 258 | 1-132 | 133-150 | 151-443 |
| 259 | 1-61 | 62-73 | 74-437 |
| 260 | 0 | 1-15 | 16-228 |
| 261 | 1-45 | 46-61 | 62-451 |
| 262 | 0 | 1-20 | 21-415 |
| 263 | 1-129 | 130-149 | 150-416 |
| 264 | 1-18 | 19-34 | 35-392 |
| 265 | 1-58 | 59-82 | 83-441 |
| 266 | 1-22 | 23-40 | 41-484 |
| 267 | 1-21 | 22-39 | 40-481 |
| 268 | 0 | 1-20 | 21-487 |
| 269 | 1-122 | 123-143 | 144-430 |
| 270 | 1-21 | 22-39 | 40-502 |
| 271 | 1-156 | 157-182 | 183-485 |
| 272 | 1-159 | 160-177 | 178-392 |
| 273 | 1-17 | 18-41 | 42-327 |
| 274 | 1-159 | 160-177 | 178-362 |
| 275 | 1-15 | 16-33 | 34-423 |
| 276 | 1-74 | 75-96 | 97-364 |
| 277 | 1-83 | 84-101 | 102-372 |
| 278 | 1-125 | 126-146 | 147-432 |
| 279 | 1-341 | 342-365 | 0 |
| 280 | 1-73 | 74-105 | 106-386 |
| 281 | 0 | 1-20 | 21-443 |
| 282 | 1-265 | 266-281 | 282-456 |
| 283 | 1-56 | 57-72 | 73-457 |
| 284 | 0 | 1-12 | 13-454 |
| 285 | 0 | 1-32 | 33-398 |
| 286 | 1-75 | 76-105 | 106-377 |
| 287 | 0 | 1-18 | 19-423 |
| 288 | 1-84 | 85-105 | 106-515 |
| 289 | 0 | 1-14 | 15-553 |
| 290 | 1-461 | 462-471 | 0 |
| 291 | 1-18 | 19-33 | 34-380 |
| 292 | 1-17 | 18-35 | 36-422 |
| 293 | 1-188 | 189-200 | 201-455 |
| 294 | 1-33 | 34-54 | 55-559 |
| 295 | 1-40 | 41-70 | 71-466 |
| 296 | 1-83 | 84-101 | 102-479 |
| 297 | 1-21 | 22-39 | 40-430 |
| 298 | 1-112 | 113-133 | 134-381 |
| 299 | 1-273 | 274-297 | 298-465 |
| 300 | 1-17 | 18-35 | 36-436 |
| 301 | 1-60 | 61-74 | 75-299 |
| 302 | 1-16 | 17-28 | 29-215 |
| 303 | 1-22 | 23-34 | 35-113 |
| 304 | 1-20 | 21-32 | 33-244 |
| 305 | 1-23 | 24-47 | 48-251 |
| 306 | 1-14 | 15-29 | 30-240 |
| 307 | 0 | 1-18 | 19-209 |
| 308 | 1-173 | 174-191 | 192-244 |
| 309 | 1-16 | 17-36 | 37-322 |
| 310 | 1-72 | 73-96 | 97-341 |
| 311 | 1-74 | 75-96 | 97-316 |
| 312 | 1-18 | 19-44 | 45-290 |
| 313 | 1-209 | 210-233 | 234-323 |
| 314 | 0 | 1-20 | 21-385 |
| 315 | 1-21 | 22-42 | 43-394 |
| 316 | 0 | 1-20 | 21-371 |
| 317 | 1-40 | 41-55 | 56-331 |
| 318 | 1-22 | 23-43 | 44-354 |
| 319 | 1-57 | 58-73 | 74-331 |
| 320 | 1-21 | 22-39 | 40-170 |
| 321 | 1-17 | 18-41 | 42-437 |
| 322 | 1-159 | 160-177 | 178-201 |
| 323 | 0 | 1-14 | 15-295 |
| 324 | 0 | 1-16 | 17-361 |
| 325 | 1-105 | 106-137 | 138-390 |
| 326 | 1-86 | 87-104 | 105-248 |
| 327 | 1-88 | 89-109 | 110-435 |
| 328 | 1-105 | 106-137 | 138-258 |
| 329 | 1-18 | 19-36 | 37-231 |
| 330 | 1-12 | 13-46 | 47-232 |
| 331 | 1-62 | 63-76 | 77-234 |
| 332 | 1-46 | 47-58 | 59-478 |
| 333 | 0 | 1-14 | 15-535 |
| 334 | 0 | 1-16 | 17-496 |
| 335 | 1-61 | 62-73 | 74-359 |
| 336 | 1-40 | 41-56 | 57-447 |
| 337 | 1-51 | 52-69 | 70-415 |
| 338 | 1-71 | 72-95 | 96-404 |
| 339 | 1-21 | 22-41 | 42-433 |
| 340 | 1-159 | 160-177 | 178-435 |
| 341 | 1-159 | 160-177 | 178-428 |
| 342 | 1-21 | 22-39 | 40-363 |
| 343 | 1-17 | 18-41 | 42-372 |
| 344 | 1-159 | 160-177 | 178-438 |
| 345 | 1-13 | 14-35 | 36-420 |
| 346 | 1-71 | 72-95 | 96-343 |
| 347 | 1-17 | 18-41 | 42-371 |
| 348 | 1-19 | 20-34 | 35-412 |
| 349 | 1-136 | 137-152 | 153-355 |
| 350 | 1-104 | 105-128 | 129-300 |
| 351 | 1-15 | 16-33 | 34-407 |
| 352 | 0 | 1-18 | 19-392 |
| 353 | 0 | 1-12 | 13-360 |
| 354 | 0 | 1-18 | 19-247 |
| 355 | 1-75 | 76-107 | 108-418 |
| 356 | 1-22 | 23-46 | 47-273 |
| 357 | 1-24 | 25-36 | 37-341 |
| 358 | 1-18 | 19-36 | 37-344 |
| 359 | 1-83 | 84-104 | 105-334 |
| 360 | 1-53 | 54-71 | 72-282 |
| 361 | 1-18 | 19-34 | 35-403 |
| 362 | 1-88 | 89-109 | 110-448 |
| 363 | 0 | 1-14 | 15-481 |
| 364 | 1-15 | 16-33 | 34-426 |
| 365 | 1-71 | 72-95 | 96-359 |
| 366 | 1-18 | 19-36 | 37-256 |
| 367 | 1-101 | 102-109 | 110-152 |
| 368 | 0 | 1-20 | 21-384 |
| 369 | 1-15 | 16-31 | 32-440 |
| 370 | 0 | 1-32 | 33-246 |
| 371 | 1-21 | 22-39 | 40-413 |
| 372 | 1-117 | 118-138 | 139-383 |
| 373 | 1-39 | 40-61 | 62-325 |
| 374 | 1-80 | 81-98 | 99-142 |
| 375 | 1-53 | 54-71 | 72-131 |
| 376 | 1-24 | 25-58 | 59-305 |
| 377 | 1-88 | 89-109 | 110-355 |
| 378 | 1-21 | 22-42 | 43-223 |
| 379 | 1-41 | 42-57 | 58-146 |
| 380 | 1-46 | 47-67 | 68-314 |
| 381 | 1-28 | 29-44 | 45-390 |
| 382 | 0 | 1-12 | 13-360 |
| 383 | 1-69 | 70-87 | 88-380 |
| 384 | 1-51 | 52-69 | 70-444 |
| 385 | 1-221 | 222-241 | 242-400 |
| 386 | 1-270 | 271-290 | 291-404 |
| 387 | 1-40 | 41-67 | 68-426 |
| 388 | 1-78 | 79-96 | 97-436 |
| 389 | 0 | 1-16 | 17-446 |
| 390 | 1-245 | 246-273 | 274-332 |
| 391 | 0 | 1-24 | 25-422 |
| 392 | 1-160 | 161-200 | 201-428 |
| 393 | 1-25 | 26-43 | 44-434 |
| 394 | 1-260 | 261-274 | 275-301 |
| 395 | 1-89 | 90-101 | 102-450 |
| 396 | 1-285 | 286-306 | 0 |
| 397 | 1-74 | 75-98 | 99-389 |
| 398 | 1-312 | 313-336 | 337-376 |
| 399 | 1-133 | 134-145 | 146-169 |
| 400 | 1-41 | 42-68 | 69-412 |
| 401 | 1-78 | 79-106 | 107-421 |
| 402 | 1-23 | 24-41 | 42-270 |
| 403 | 1-74 | 75-104 | 105-343 |
| 404 | 1-28 | 29-40 | 41-350 |
| 405 | 1-81 | 82-101 | 102-386 |
| 406 | 0 | 1-18 | 19-381 |
| 407 | 1-31 | 32-53 | 54-367 |
| 408 | 0 | 1-12 | 13-152 |
| 409 | 1-41 | 42-61 | 62-432 |
| 410 | 0 | 1-12 | 13-349 |
| 411 | 1-16 | 17-32 | 33-385 |
| 412 | 1-129 | 130-143 | 144-375 |
| 413 | 1-10 | 11-22 | 23-180 |
| 414 | 0 | 1-20 | 21-374 |
| 415 | 1-52 | 53-66 | 67-404 |
| 416 | 0 | 1-18 | 19-377 |
| 417 | 0 | 1-12 | 13-243 |
| 418 | 1-41 | 42-61 | 62-313 |
| 419 | 1-25 | 26-53 | 54-334 |
| 420 | 1-24 | 25-44 | 45-396 |
| 421 | 1-209 | 210-221 | 222-375 |
| 422 | 1-25 | 26-45 | 46-307 |
| 423 | 1-37 | 38-55 | 56-304 |
| 424 | 1-123 | 124-147 | 148-267 |
| 425 | 1-43 | 44-61 | 62-405 |
| 426 | 1-22 | 23-47 | 48-363 |
| 427 | 1-277 | 278-291 | 0 |
| 428 | 1-25 | 26-53 | 54-258 |
| 429 | 1-21 | 22-42 | 43-438 |
| 430 | 1-69 | 70-87 | 88-374 |
| 431 | 1-38 | 39-54 | 55-110 |
| 432 | 0 | 1-16 | 17-311 |
| 433 | 0 | 1-14 | 15-300 |
| 434 | 1-89 | 90-113 | 114-237 |
| 435 | 1-27 | 28-36 | 37-264 |
| 436 | 1-182 | 183-196 | 197-206 |
| 437 | 1-192 | 193-222 | 223-261 |
| 438 | 1-79 | 80-100 | 101-341 |
| 439 | 1-12 | 13-58 | 9-339 |
| 440 | 1-60 | 61-81 | 82-360 |
| 441 | 0 | 1-24 | 25-376 |
| 442 | 1-53 | 54-68 | 69-301 |
| 443 | 1-119 | 120-131 | 132-310 |
| 444 | 1-60 | 61-76 | 77-241 |
| 445 | 1-25 | 26-52 | 53-352 |
| 446 | 0 | 1-26 | 27-313 |
| 447 | 1-68 | 69-90 | 91-402 |
| 448 | 1-39 | 40-63 | 64-418 |
| 449 | 1-88 | 89-108 | 109-414 |
| 450 | 1-61 | 62-79 | 80-364 |
| 451 | 1-12 | 13-26 | 27-319 |
| 452 | 1-77 | 78-93 | 94-253 |
| 453 | 0 | 1-26 | 27-407 |
| 454 | 1-53 | 54-73 | 74-397 |
| 455 | 1-57 | 58-73 | 74-403 |
| 456 | 0 | 1-24 | 25-297 |
| 457 | 1-20 | 21-58 | 59-451 |
| 458 | 1-32 | 33-50 | 51-211 |
| 459 | 1-12 | 13-36 | 37-397 |
| 460 | 1-46 | 47-60 | 61-347 |
| 461 | 1-95 | 96-115 | 116-351 |
| 462 | 0 | 1-22 | 23-88 |
| 463 | 1-252 | 253-270 | 271-355 |
| 464 | 1-82 | 83-96 | 97-360 |
| 465 | 1-35 | 36-71 | 72-342 |
| 466 | 0 | 1-12 | 13-361 |
| 467 | 1-10 | 11-34 | 35-428 |
| 468 | 1-322 | 323-343 | 344-414 |
| 469 | 0 | 1-12 | 13-268 |
| 470 | 1-114 | 115-138 | 139-377 |
| 471 | 0 | 1-20 | 21-411 |
| 472 | 1-25 | 26-53 | 54-388 |
| 473 | 1-20 | 21-38 | 39-338 |
| 474 | 0 | 1-18 | 19-347 |
| 475 | 1-30 | 31-40 | 41-401 |
| 476 | 1-30 | 31-50 | 51-394 |
| 477 | 1-186 | 187-216 | 217-392 |
| 478 | 1-100 | 101-124 | 125-434 |
| 479 | 1-34 | 35-44 | 45-438 |
| 480 | 0 | 1-18 | 19-386 |
| 481 | 1-16 | 17-30 | 31-382 |
| 482 | 1-23 | 24-41 | 42-480 |
| 483 | 1-48 | 49-66 | 67-437 |
| 484 | 0 | 1-14 | 15-109 |
| 485 | 1-27 | 28-45 | 46-410 |
| 486 | 1-285 | 286-306 | 0 |
| 487 | 1-40 | 41-56 | 57-327 |
| 488 | 0 | 1-10 | 11-441 |
| 489 | 0 | 1-16 | 17-355 |
| 490 | 1-425 | 426-437 | 0 |
| 491 | 1-99 | 100-123 | 124-338 |
| 492 | 1-13 | 14-29 | 30-413 |
| 493 | 1-368 | 369-384 | 385-415 |
| 494 | 1-34 | 35-64 | 65-384 |
| 495 | 1-31 | 32-57 | 58-433 |
| 496 | 1-55 | 56-75 | 76-135 |
| 497 | 0 | 1-22 | 23-415 |
| 498 | 1-396 | 397-406 | 0 |
| 499 | 1-102 | 103-114 | 115-411 |
| 500 | 0 | 1-12 | 13-345 |
| 501 | 0 | 1-22 | 23-358 |
| 502 | 0 | 1-20 | 21-373 |
| 503 | 0 | 1-14 | 15-363 |
| 504 | 0 | 1-27 | 28-364 |
| 505 | 0 | 1-36 | 37-408 |
| 506 | 1-49 | 50-67 | 68-372 |
| 507 | 1-34 | 35-50 | 51-330 |
| 508 | 0 | 1-12 | 13-308 |
| 509 | 1-25 | 26-37 | 38-385 |
| 510 | 1-97 | 98-109 | 110-249 |
| 511 | 1-25 | 26-46 | 47-260 |
| 512 | 1-93 | 94-119 | 120-365 |
| 513 | 1-75 | 76-105 | 106-284 |
| 514 | 1-40 | 41-68 | 69-314 |
| 515 | 1-41 | 42-69 | 70-343 |
| 516 | 1-180 | 181-192 | 193-265 |
| 517 | 1-34 | 35-56 | 57-405 |
| 518 | 1-91 | 92-115 | 116-367 |
| 519 | 1-31 | 32-53 | 54-386 |
| 520 | 1-53 | 54-67 | 68-228 |
| 521 | 1-10 | 11-28 | 29-445 |
| 522 | 1-30 | 31-44 | 45-338 |
| 523 | 1-127 | 128-157 | 158-327 |
| 524 | 1-22 | 23-40 | 41-305 |
| 525 | 1-10 | 11-38 | 39-241 |
| 526 | 1-117 | 118-138 | 139-195 |
| 527 | 0 | 1-20 | 21-237 |
| 528 | 0 | 1-16 | 17-281 |
| 529 | 0 | 1-14 | 15-263 |
| 530 | 1-21 | 22-33 | 34-372 |
| 531 | 1-88 | 89-109 | 110-299 |
| 532 | 1-145 | 146-166 | 167-321 |
| 533 | 1-35 | 36-67 | 68-477 |
| 534 | 1-32 | 33-46 | 47-406 |
| 535 | 0 | 1-12 | 13-457 |
| 536 | 1-97 | 98-107 | 108-350 |
| 537 | 1-159 | 160-180 | 181-373 |
| 538 | 1-29 | 30-55 | 56-264 |
| 539 | 1-20 | 21-36 | 37-349 |
| 540 | 1-21 | 22-33 | 34-409 |
| 541 | 0 | 1-14 | 15-348 |
| 542 | 1-93 | 94-105 | 106-378 |
| 543 | 1-33 | 34-49 | 50-398 |
| 544 | 0 | 1-12 | 13-366 |
| 545 | 1-17 | 18-31 | 32-338 |
| 546 | 1-86 | 87-102 | 103-238 |
| 547 | 1-67 | 68-79 | 80-248 |
| 548 | 0 | 1-18 | 19-357 |
| 549 | 0 | 1-18 | 19-418 |
| 550 | 1-33 | 34-61 | 62-445 |
| 551 | 1-14 | 15-44 | 45-433 |
| 552 | 1-43 | 44-70 | 71-342 |
| 553 | 1-29 | 30-56 | 57-167 |
| 554 | 1-12 | 13-26 | 27-434 |
| 555 | 1-23 | 24-43 | 44-281 |
| 556 | 1-133 | 134-148 | 149-244 |
| 557 | 0 | 1-21 | 22-270 |
| 558 | 1-250 | 251-278 | 279-346 |
| 559 | 1-33 | 34-53 | 54-420 |
| 560 | 1-38 | 39-53 | 54-233 |
| 561 | 1-27 | 28-48 | 49-238 |
| 562 | 1-19 | 20-43 | 44-279 |
| 563 | 0 | 1-18 | 19-264 |
| 564 | 1-62 | 63-80 | 81-312 |
| 565 | 1-101 | 102-125 | 126-281 |
| 566 | 1-307 | 308-334 | 335-394 |
| 567 | 1-153 | 154-183 | 184-320 |
| 568 | 1-30 | 31-44 | 45-297 |
| 569 | 1-31 | 32-49 | 50-141 |
| 570 | 1-96 | 97-114 | 115-446 |
| 571 | 1-21 | 22-33 | 34-142 |
| 572 | 1-135 | 136-149 | 150-347 |
| 573 | 1-38 | 39-52 | 53-325 |
| 574 | 0 | 1-14 | 15-364 |
| 575 | 1-76 | 77-97 | 98-344 |
| 576 | 1-220 | 221-250 | 251-317 |
| 577 | 0 | 1-22 | 23-316 |
| 578 | 1-326 | 327-340 | 341-428 |
| 579 | 1-70 | 71-109 | 110-191 |
| 580 | 0 | 1-18 | 19-314 |
| 581 | 0 | 1-16 | 17-346 |
| 582 | 1-181 | 182-207 | 208-269 |
| 583 | 1-183 | 184-205 | 206-366 |
| 584 | 1-220 | 221-228 | 229-383 |
| 585 | 1-64 | 65-84 | 85-281 |
| 586 | 0 | 1-22 | 23-270 |
| 587 | 1-17 | 18-47 | 48-232 |
| 588 | 1-18 | 19-46 | 47-346 |
| 589 | 1-71 | 72-85 | 86-331 |
| 590 | 1-287 | 288-301 | 302-355 |
| 591 | 1-13 | 14-27 | 28-409 |
| 592 | 1-40 | 41-56 | 57-370 |
| 593 | 1-26 | 27-44 | 45-383 |
| 594 | 0 | 1-24 | 25-166 |
| 595 | 0 | 1-12 | 13-349 |
| 596 | 1-158 | 159-194 | 195-205 |
| 597 | 1-316 | 317-337 | 338-379 |
| 598 | 0 | 1-26 | 27-390 |
| 599 | 1-59 | 60-89 | 90-317 |
| 600 | 0 | 1-22 | 23-321 |
| 601 | 1-146 | 147-170 | 171-339 |
| 602 | 1-61 | 62-70 | 71-327 |
| 603 | 1-100 | 101-121 | 122-336 |
| 604 | 1-17 | 18-51 | 52-354 |
| 605 | 1-65 | 66-83 | 84-359 |
| 606 | 0 | 1-28 | 29-400 |
| 607 | 1-13 | 14-29 | 30-276 |
| 608 | 1-83 | 84-104 | 105-388 |
| 609 | 1-53 | 54-79 | 80-452 |
| 610 | 0 | 1-14 | 15-368 |
| 611 | 1-21 | 22-45 | 46-315 |
| 612 | 0 | 1-20 | 21-367 |
| 613 | 1-40 | 41-56 | 57-255 |
| 614 | 0 | 1-12 | 13-223 |
| 615 | 1-44 | 45-56 | 57-307 |
| 616 | 1-27 | 28-39 | 40-321 |
| 617 | 1-368 | 369-384 | 385-426 |
| 618 | 0 | 1-16 | 17-343 |
| 619 | 1-252 | 253-278 | 279-428 |
| 620 | 1-66 | 67-86 | 87-395 |
| 621 | 1-83 | 84-99 | 100-395 |
| 622 | 1-11 | 12-29 | 30-446 |
| 623 | 1-185 | 186-199 | 200-460 |
| 624 | 1-71 | 72-83 | 84-401 |
| 625 | 0 | 1-14 | 15-467 |
| 626 | 1-34 | 35-52 | 53-369 |
| 627 | 1-57 | 58-71 | 72-286 |
| 628 | 1-102 | 103-120 | 121-372 |
| 629 | 1-350 | 351-386 | 387-413 |
| 630 | 1-85 | 86-101 | 102-385 |
| 631 | 1-323 | 324-335 | 336-407 |
| 632 | 1-50 | 51-77 | 78-285 |
| 633 | 1-80 | 81-96 | 97-232 |
| 634 | 0 | 1-34 | 35-458 |
| 635 | 1-191 | 192-203 | 204-423 |
| 636 | 1-18 | 19-30 | 31-357 |
| 637 | 0 | 1-30 | 31-439 |
| 638 | 1-87 | 88-103 | 104-445 |
| 639 | 1-165 | 166-177 | 178-355 |
| 640 | 1-73 | 74-89 | 90-421 |
| 641 | 1-41 | 42-55 | 56-359 |
| 642 | 0 | 1-24 | 25-196 |
| 643 | 1-224 | 225-238 | 239-411 |
| 644 | 1-54 | 55-80 | 81-264 |
| 645 | 1-355 | 356-373 | 374-397 |
| 646 | 1-142 | 143-158 | 159-329 |
| 647 | 1-40 | 41-72 | 73-365 |
| 648 | 1-98 | 99-110 | 111-365 |
| 649 | 1-26 | 27-58 | 59-401 |
| 650 | 1-124 | 125-138 | 139-471 |
| 651 | 0 | 1-12 | 13-509 |
| 652 | 1-78 | 79-99 | 100-382 |
| 653 | 1-14 | 15-26 | 27-461 |
| 654 | 1-50 | 51-77 | 78-468 |
| 655 | 1-50 | 51-77 | 78-559 |
| 656 | 1-363 | 364-387 | 388-465 |
| 657 | 1-29 | 30-43 | 44-280 |
| 658 | 1-62 | 63-78 | 79-137 |
| 659 | 1-212 | 213-227 | 228-323 |
| 660 | 1-43 | 44-70 | 71-386 |
| 661 | 1-69 | 70-102 | 103-429 |
| 662 | 1-423 | 424-437 | 0 |
| 663 | 0 | 1-38 | 39-369 |
| 664 | 1-26 | 27-46 | 47-397 |
| 665 | 1-38 | 39-58 | 59-349 |
| 666 | 1-16 | 17-28 | 29-438 |
| 667 | 1-38 | 39-65 | 66-289 |
| 668 | 0 | 1-16 | 17-263 |
| 669 | 1-40 | 41-54 | 55-414 |
| 670 | 1-43 | 44-61 | 62-218 |
| 671 | 0 | 1-28 | 29-461 |
| 672 | 0 | 1-18 | 19-315 |
| 673 | 1-127 | 128-151 | 152-347 |
| 674 | 1-89 | 90-113 | 114-436 |
| 675 | 1-25 | 26-53 | 54-376 |
| 676 | 0 | 1-12 | 13-333 |
| 677 | 1-19 | 20-39 | 40-415 |
| 678 | 0 | 1-18 | 19-366 |
| 679 | 1-294 | 295-314 | 315-343 |
| 680 | 0 | 1-24 | 25-350 |
| 681 | 1-197 | 198-209 | 210-337 |
| 682 | 1-17 | 18-29 | 30-406 |
| 683 | 1-125 | 126-143 | 144-389 |
| 684 | 1-18 | 19-42 | 43-314 |
| 685 | 1-27 | 28-54 | 55-416 |
| 686 | 1-14 | 15-28 | 29-165 |
| 687 | 1-184 | 185-202 | 203-245 |
| 688 | 1-83 | 84-93 | 94-374 |
| 689 | 1-15 | 16-33 | 34-389 |
| 690 | 0 | 1-14 | 15-367 |
| 691 | 1-99 | 100-107 | 108-289 |
| 692 | 1-22 | 23-40 | 41-367 |
| 693 | 1-165 | 166-181 | 182-401 |
| 694 | 1-90 | 91-111 | 112-403 |
| 695 | 1-23 | 24-33 | 34-205 |
| 696 | 1-181 | 182-197 | 198-228 |
| 697 | 1-97 | 98-121 | 122-286 |
| 698 | 1-72 | 73-86 | 87-222 |
| 699 | 1-39 | 40-57 | 58-313 |
| 700 | 1-31 | 32-53 | 54-288 |
| 701 | 1-52 | 53-66 | 67-233 |
| 702 | 0 | 1-10 | 11-291 |
| 703 | 1-30 | 31-46 | 47-299 |
| 704 | 1-93 | 94-115 | 116-378 |
| 705 | 1-94 | 95-114 | 115-348 |
| 706 | 1-58 | 59-76 | 77-302 |
| 707 | 0 | 1-22 | 23-282 |
| 708 | 1-27 | 28-39 | 40-269 |
| 709 | 1-41 | 42-55 | 56-330 |
| 710 | 1-23 | 24-49 | 50-360 |
| 711 | 1-95 | 96-107 | 108-288 |
| 712 | 1-31 | 32-49 | 50-207 |
| 713 | 0 | 1-24 | 25-351 |
| 714 | 1-31 | 32-49 | 50-354 |
| 715 | 1-14 | 15-34 | 35-280 |
| 716 | 1-14 | 15-26 | 27-338 |
| 717 | 1-68 | 69-107 | 108-390 |
| 718 | 0 | 1-12 | 13-251 |
| 719 | 0 | 1-16 | 17-349 |
| 720 | 1-41 | 42-57 | 58-350 |
| 721 | 1-155 | 156-173 | 174-339 |
| 722 | 1-26 | 27-54 | 55-323 |
| 723 | 1-148 | 149-166 | 167-383 |
| 724 | 1-39 | 40-53 | 54-393 |
| 725 | 1-32 | 33-56 | 57-373 |
| 726 | 1-52 | 53-66 | 67-378 |
| 727 | 0 | 1-16 | 17-346 |
| 728 | 1-30 | 31-44 | 45-380 |
| 729 | 1-284 | 285-305 | 306-397 |
| 730 | 1-80 | 81-101 | 102-381 |
| 731 | 0 | 1-14 | 15-172 |
| 732 | 1-150 | 151-174 | 175-319 |
| 733 | 1-57 | 58-75 | 76-344 |
| 734 | 1-222 | 223-238 | 239-383 |
| 735 | 0 | 1-10 | 11-340 |
| 736 | 1-67 | 68-87 | 88-406 |
| 737 | 0 | 1-14 | 15-375 |
| 738 | 1-81 | 82-105 | 106-412 |
| 739 | 1-247 | 248-277 | 278-416 |
| 740 | 1-21 | 22-41 | 42-418 |
| 741 | 1-11 | 12-38 | 39-302 |
| 742 | 0 | 1-12 | 13-431 |
| 743 | 1-13 | 14-27 | 28-247 |
| 744 | 1-102 | 103-126 | 127-344 |
| 745 | 0 | 1-20 | 21-309 |
| 746 | 1-25 | 26-37 | 38-320 |
| 747 | 1-89 | 90-113 | 114-352 |
| 748 | 0 | 1-18 | 19-294 |
| 749 | 1-106 | 107-120 | 121-396 |
| 750 | 1-115 | 116-137 | 138-304 |
| 751 | 1-261 | 262-279 | 280-372 |
| 752 | 1-265 | 266-271 | 272-385 |
| 753 | 0 | 1-20 | 21-257 |
| 754 | 1-89 | 90-113 | 114-378 |
| 755 | 1-19 | 20-31 | 32-398 |
| 756 | 1-15 | 16-31 | 32-223 |
| 757 | 1-81 | 82-93 | 94-346 |
| 758 | 0 | 1-36 | 37-405 |
| 759 | 1-21 | 22-48 | 49-390 |
| 760 | 1-42 | 43-60 | 61-394 |
| 761 | 1-39 | 40-57 | 58-234 |
| 762 | 0 | 1-16 | 17-370 |
| 763 | 0 | 1-12 | 13-372 |
| 764 | 1-35 | 36-45 | 46-212 |
| 765 | 1-27 | 28-45 | 46-356 |
| 766 | 1-158 | 159-174 | 175-318 |
| 767 | 1-210 | 211-226 | 227-373 |
| 768 | 1-30 | 31-51 | 52-256 |
| 769 | 1-45 | 46-65 | 66-378 |
| 770 | 1-16 | 17-68 | 69-328 |
| 771 | 1-24 | 25-46 | 47-381 |
| 772 | 1-147 | 148-161 | 162-366 |
| 773 | 1-99 | 100-111 | 112-302 |
| 774 | 1-21 | 22-39 | 40-346 |
| 775 | 1-70 | 71-82 | 83-392 |
| 776 | 1-32 | 33-46 | 47-333 |
| 777 | 1-230 | 231-252 | 253-391 |
| 778 | 1-64 | 65-88 | 89-268 |
| 779 | 1-43 | 44-71 | 72-436 |
| 780 | 1-15 | 16-53 | 54-358 |
| 782 | 1-368 | 369-376 | 377-386 |
| 783 | 1-82 | 83-103 | 104-326 |
| 784 | 1-24 | 25-40 | 41-278 |
| 785 | 1-99 | 100-117 | 118-298 |
| 786 | 1-55 | 56-71 | 72-356 |
| 787 | 0 | 1-18 | 19-361 |
| 788 | 1-19 | 20-49 | 50-326 |
| 789 | 1-12 | 13-36 | 37-265 |
| 790 | 1-4 | 5-20 | 21-278 |
| 791 | 1-14 | 15-44 | 45-307 |
| 792 | 1-61 | 62-79 | 80-386 |
| 793 | 1-42 | 43-54 | 55-266 |
| 794 | 1-199 | 200-211 | 0 |
| 795 | 1-28 | 29-46 | 47-320 |
| 796 | 1-54 | 55-80 | 81-256 |
| 797 | 1-14 | 15-28 | 29-147 |
| 798 | 0 | 1-12 | 13-350 |
| 799 | 1-74 | 75-104 | 105-499 |
| 800 | 0 | 1-22 | 23-388 |
| 801 | 1-47 | 48-63 | 64-378 |
| 802 | 1-124 | 125-154 | 155-244 |
| 803 | 1-12 | 13-26 | 27-464 |
| 804 | 0 | 1-34 | 35-326 |
| 805 | 0 | 1-16 | 17-291 |
| 806 | 1-21 | 22-33 | 34-228 |
| 807 | 1-80 | 81-98 | 99-178 |
| 808 | 1-87 | 88-107 | 108-388 |
| 809 | 1-57 | 58-87 | 88-438 |
| 810 | 1-29 | 30-41 | 42-509 |
| 811 | 1-17 | 18-51 | 52-270 |
| 812 | 0 | 1-22 | 23-280 |
| 813 | 0 | 1-12 | 13-220 |
| 814 | 1-32 | 33-52 | 53-353 |
| 815 | 1-64 | 65-76 | 77-321 |
| 816 | 1-104 | 105-116 | 117-369 |
| 817 | 0 | 1-12 | 13-276 |
| 818 | 1-35 | 36-47 | 48-278 |
| 819 | 1-15 | 16-33 | 34-166 |
| 820 | 0 | 1-20 | 21-268 |
| 821 | 1-32 | 33-53 | 54-251 |
| 822 | 1-222 | 223-243 | 244-280 |
| 823 | 1-24 | 25-51 | 52-306 |
| 824 | 1-74 | 75-98 | 99-135 |
| 825 | 0 | 1-24 | 25-356 |
| 826 | 0 | 1-24 | 25-214 |
| 827 | 1-155 | 156-167 | 0 |
| 828 | 1-11 | 12-31 | 32-328 |
| 829 | 1-32 | 33-46 | 47-415 |
| 830 | 1-43 | 44-59 | 60-285 |
| 831 | 1-46 | 47-60 | 61-212 |
| 832 | 1-82 | 83-115 | 116-208 |
| 833 | 0 | 1-16 | 17-181 |
| 834 | 1-10 | 11-16 | 17-127 |
| 835 | 1-21 | 22-41 | 42-187 |
| 836 | 1-54 | 55-62 | 63-485 |
| 837 | 1-11 | 12-29 | 30-456 |
| 838 | 1-85 | 86-111 | 112-406 |
| 839 | 0 | 1-18 | 19-262 |
| 840 | 1-396 | 397-406 | 0 |
| 841 | 1-98 | 99-116 | 117-144 |
| 842 | 1-49 | 50-76 | 77-262 |
| 843 | 1-167 | 168-191 | 192-438 |
| 844 | 0 | 1-12 | 13-277 |
| 845 | 1-55 | 56-71 | 72-459 |
| 846 | 1-59 | 60-85 | 86-266 |
| 847 | 1-51 | 52-72 | 73-337 |
| 848 | 1-50 | 51-72 | 73-315 |
| 849 | 1-63 | 64-87 | 88-129 |
| 850 | 0 | 1-12 | 13-436 |
| 851 | 1-13 | 14-29 | 30-247 |
| 852 | 0 | 1-20 | 21-385 |
| 853 | 1-63 | 64-75 | 76-422 |
| 854 | 1-238 | 239-259 | 260-270 |
| 855 | 1-15 | 16-33 | 34-317 |
| 856 | 0 | 1-18 | 19-351 |
| 857 | 1-154 | 155-172 | 173-324 |
| 858 | 1-46 | 47-60 | 61-273 |
| 859 | 1-86 | 87-102 | 103-356 |
| 860 | 1-82 | 83-106 | 107-375 |
| 861 | 1-16 | 17-50 | 51-357 |
| 862 | 1-94 | 95-106 | 107-495 |
| 863 | 0 | 1-14 | 15-458 |
| 864 | 1-91 | 92-111 | 112-235 |
| 865 | 0 | 1-16 | 17-418 |
| 866 | 0 | 1-32 | 33-417 |
| 867 | 1-115 | 116-135 | 136-493 |
| 868 | 0 | 1-22 | 23-487 |
| 869 | 1-186 | 187-207 | 208-385 |
| 870 | 0 | 1-12 | 13-438 |
| 871 | 0 | 1-24 | 25-520 |
| 872 | 1-106 | 107-121 | 122-207 |
| 873 | 1-50 | 51-77 | 78-475 |
| 874 | 0 | 1-34 | 35-337 |
| 875 | 1-60 | 61-78 | 79-245 |
| 876 | 1-28 | 29-42 | 43-345 |
| 877 | 1-125 | 126-137 | 138-227 |
| 878 | 1-21 | 22-43 | 44-354 |
| 879 | 1-401 | 402-411 | 0 |
| 880 | 1-98 | 99-110 | 111-445 |
| 881 | 1-151 | 152-165 | 166-192 |
| 882 | 1-86 | 87-102 | 103-455 |
| 883 | 1-18 | 19-39 | 40-151 |
| 884 | 0 | 1-8 | 9-388 |
| 885 | 1-29 | 30-47 | 48-341 |
| 886 | 0 | 1-12 | 13-261 |
| 887 | 0 | 1-90 | 91-241 |
| 888 | 1-50 | 51-77 | 78-393 |
| 889 | 0 | 1-16 | 17-332 |
| 890 | 1-40 | 41-67 | 68-417 |
| 891 | 1-99 | 100-111 | 112-384 |
| 892 | 1-15 | 16-45 | 46-396 |
| 893 | 1-149 | 150-185 | 186-362 |
| 894 | 1-359 | 360-377 | 378-458 |
| 895 | 1-42 | 43-69 | 70-416 |
| 896 | 0 | 1-14 | 15-342 |
| 897 | 1-14 | 15-35 | 36-399 |
| 898 | 1-82 | 83-98 | 99-404 |
| 899 | 0 | 1-14 | 15-342 |
| 900 | 1-10 | 11-26 | 27-317 |
| 901 | 1-29 | 30-47 | 48-297 |
| 902 | 1-119 | 120-140 | 141-296 |
| 903 | 1-13 | 14-39 | 40-419 |
| 904 | 1-23 | 24-51 | 52-392 |
| 905 | 1-30 | 31-44 | 45-129 |
| 906 | 1-18 | 19-44 | 45-255 |
| 907 | 1-198 | 199-210 | 211-227 |
| 908 | 1-104 | 105-126 | 127-413 |
| 909 | 0 | 1-14 | 15-452 |
| 910 | 1-74 | 75-104 | 105-403 |
| 911 | 1-74 | 75-95 | 96-374 |
| 912 | 1-92 | 93-116 | 117-381 |
| 913 | 1-92 | 93-116 | 117-375 |
| 914 | 1-86 | 87-102 | 103-202 |
| 915 | 0 | 1-16 | 17-385 |
| 916 | 1-92 | 93-116 | 117-384 |
| 917 | 1-216 | 217-224 | 225-364 |
| 918 | 0 | 1-14 | 15-377 |
| 919 | 1-37 | 38-49 | 50-310 |
| 920 | 1-25 | 26-37 | 38-276 |
| 921 | 1-20 | 21-38 | 39-253 |
| 922 | 1-71 | 72-85 | 86-361 |
| 923 | 0 | 1-16 | 17-325 |
| 924 | 1-30 | 31-42 | 43-411 |
| 925 | 0 | 1-34 | 35-210 |
| 926 | 0 | 1-27 | 28-159 |
| 927 | 1-14 | 15-26 | 27-145 |
| 928 | 0 | 1-14 | 15-165 |
| 929 | 1-21 | 22-39 | 40-338 |
| 930 | 1-52 | 53-66 | 67-345 |
| 931 | 0 | 1-8 | 9-352 |
| 932 | 1-197 | 198-209 | 210-295 |
| 933 | 1-41 | 42-55 | 56-124 |
| 934 | 1-10 | 11-24 | 25-359 |
| 935 | 1-146 | 147-170 | 171-391 |
| 936 | 1-30 | 31-44 | 45-356 |
| 937 | 0 | 1-28 | 29-408 |
| 938 | 1-14 | 15-44 | 45-409 |
| 939 | 0 | 1-20 | 21-286 |
| 940 | 1-86 | 87-110 | 111-277 |
| 941 | 1-186 | 187-222 | 223-384 |
| 942 | 1-86 | 87-102 | 103-302 |
| 943 | 0 | 1-26 | 27-302 |
| 944 | 1-26 | 27-38 | 39-370 |
| 945 | 1-94 | 95-120 | 121-205 |
| 946 | 1-40 | 41-68 | 69-395 |
| 947 | 1-29 | 30-53 | 54-386 |
| 948 | 1-92 | 93-116 | 117-388 |
| 949 | 0 | 1-12 | 13-366 |
| 950 | 1-18 | 19-46 | 47-365 |
| 951 | 1-15 | 16-53 | 54-352 |
| 952 | 1-33 | 34-47 | 48-120 |
| 953 | 1-64 | 65-84 | 85-250 |
| 954 | 1-98 | 99-110 | 111-342 |
| 955 | 1-50 | 51-77 | 78-366 |
| 956 | 1-88 | 89-115 | 116-456 |
| 957 | 1-52 | 53-78 | 79-392 |
| 958 | 1-9 | 10-33 | 34-202 |
| 959 | 1-18 | 19-44 | 45-229 |
| 960 | 0 | 1-20 | 21-386 |
| 961 | 1-31 | 32-49 | 50-240 |
| 962 | 1-25 | 26-53 | 54-386 |
| 963 | 1-293 | 294-307 | 308-318 |
| 964 | 1-310 | 311-324 | 325-388 |
| 965 | 1-53 | 54-73 | 74-172 |
| 966 | 1-44 | 45-62 | 63-449 |
| 967 | 1-98 | 99-116 | 117-419 |
| 968 | 1-50 | 51-77 | 78-268 |
| 969 | 1-21 | 22-43 | 44-399 |
| 970 | 0 | 1-18 | 19-295 |
| 971 | 0 | 1-14 | 15-313 |
| 972 | 1-54 | 55-72 | 73-153 |
| 973 | 1-48 | 49-62 | 63-127 |
| 974 | 1-46 | 47-61 | 62-271 |
| 975 | 1-51 | 52-65 | 66-254 |
| 976 | 1-60 | 61-96 | 97-180 |
| 977 | 1-54 | 55-72 | 73-308 |
| 978 | 0 | 1-18 | 19-248 |
| 979 | 1-15 | 16-27 | 28-253 |
| 980 | 1-76 | 77-102 | 103-206 |
| 981 | 1-21 | 22-51 | 52-231 |
| 982 | 0 | 1-14 | 15-241 |
| 983 | 1-42 | 43-57 | 58-181 |
| 984 | 1-37 | 38-49 | 50-213 |
| 985 | 1-10 | 11-24 | 25-200 |
| 986 | 1-59 | 60-75 | 76-223 |
| 987 | 1-87 | 88-101 | 102-340 |
| 988 | 1-347 | 348-375 | 376-484 |
| 989 | 1-131 | 132-158 | 159-492 |
| 990 | 1-14 | 15-30 | 31-303 |
| 991 | 1-221 | 222-237 | 238-450 |
| 992 | 1-63 | 64-96 | 97-415 |
| 993 | 1-137 | 138-158 | 159-351 |
| 994 | 1-60 | 61-74 | 75-130 |
| 995 | 1-22 | 23-34 | 35-402 |
| 996 | 1-250 | 251-277 | 278-381 |
| 997 | 1-114 | 115-132 | 133-363 |
| 998 | 1-92 | 93-116 | 117-362 |
| 999 | 1-98 | 99-128 | 129-383 |
| 1000 | 1-20 | 21-44 | 45-356 |
| 1001 | 1-68 | 69-88 | 89-563 |
| 1002 | 0 | 1-18 | 19-567 |
| 1003 | 0 | 1-18 | 19-477 |
| 1004 | 1-15 | 16-33 | 34-606 |
| 1005 | 1-529 | 530-555 | 556-577 |
| 1006 | 0 | 1-12 | 13-503 |
| 1007 | 1-213 | 214-237 | 238-391 |
| 1008 | 1-170 | 171-188 | 189-355 |
| 1009 | 1-223 | 224-247 | 248-341 |
| 1010 | 1-12 | 13-34 | 35-212 |
| 1011 | 1-17 | 18-43 | 44-300 |
| 1012 | 1-234 | 235-260 | 261-400 |
| 1013 | 1-23 | 24-39 | 40-431 |
| 1014 | 1-144 | 145-168 | 169-317 |
| 1015 | 0 | 1-12 | 13-344 |
| 1016 | 1-286 | 287-300 | 301-469 |
| 1017 | 1-127 | 128-145 | 146-230 |
| 1018 | 1-127 | 128-155 | 156-317 |
| 1019 | 1-132 | 133-150 | 151-413 |
| 1020 | 1-182 | 183-212 | 213-533 |
| 1021 | 1-453 | 454-491 | 0 |
| 1022 | 1-70 | 71-82 | 83-358 |
| 1023 | 1-13 | 14-31 | 32-443 |
| 1024 | 1-256 | 257-286 | 287-392 |
| 1025 | 1-67 | 68-79 | 80-156 |
| 1026 | 1-201 | 202-241 | 242-333 |
| 1027 | 1-77 | 78-89 | 90-376 |
| 1028 | 1-16 | 17-36 | 37-439 |
| 1029 | 1-283 | 284-321 | 322-359 |
| 1030 | 1-18 | 19-34 | 35-426 |
| 1031 | 1-95 | 96-113 | 114-353 |
| 1032 | 1-164 | 165-176 | 177-439 |
| 1033 | 1-23 | 24-41 | 42-314 |
| 1034 | 1-19 | 20-31 | 32-199 |
| 1035 | 1-21 | 22-39 | 40-445 |
| 1036 | 1-120 | 121-138 | 139-457 |
| 1037 | 1-192 | 193-204 | 205-408 |
| 1038 | 1-334 | 335-346 | 347-401 |
| 1039 | 1-101 | 102-113 | 114-349 |
| 1040 | 1-17 | 18-33 | 34-256 |
| 1041 | 1-161 | 162-191 | 192-312 |
| 1042 | 0 | 1-14 | 15-338 |
| 1043 | 1-72 | 73-94 | 95-352 |
| 1044 | 1-106 | 107-115 | 116-306 |
| 1045 | 1-247 | 248-277 | 278-327 |
| 1046 | 1-217 | 218-229 | 230-326 |
| 1047 | 1-196 | 197-208 | 209-421 |
| 1048 | 1-332 | 333-348 | 349-371 |
| 1049 | 0 | 1-12 | 13-266 |
| 1050 | 1-149 | 150-175 | 176-355 |
| 1051 | 1-117 | 118-135 | 136-228 |
| 1052 | 1-279 | 280-297 | 298-445 |
| 1053 | 1-363 | 364-381 | 382-418 |
| 1054 | 0 | 1-18 | 19-417 |

The present invention also provides novel microsatellites, which have not been previously been known to occur in tandem repeats in natural DNA, based on BLASTN similarity searches using at least three repeats for similarity search against the EMBL DNA database. Isolated polynucleotides are thus provided which comprise at least three repeats of a sequence provided in SEQ ID NO: 1055-1057.

The isolated polynucleotide sequences of the present invention have utility in the detection of DNA polymorphisms, in genome mapping, in physical mapping and positional cloning of genes, in variety identification, and in evaluation of genetic variability within and between plant tissues, populations, cultivars, species and species groups. More specifically, the inventive polynucleotide sequences may be used to design hybridization probes for oligonucleotide fingerprinting and library screening, and to design primers for microsatellite-primed PCR, as detailed below.

Microsatellites are highly useful as molecular markers for genetic mapping, population genetic analysis, strain identification and plant breeding. The isolated microsatellite repeats with their single copy flanking sequences provide locus-specific markers. The flanking sequences may be used to design locus-specific oligonucleotide primers to amplify and detect the presence of the microsatellite sequence in a plant's genome.

Most microsatellites are not subject to selection pressures and undergo high rates of mutation which generate extensive allelic variation and high levels of heterozygosity. The use of these hypervariable microsatellite markers may uncover genetic diversity in populations that exhibit low levels of variability of other markers (e.g., allozymes and mitochondrial DNA).

The size, abundance and comparatively random distribution of microsatellite sequences in eucaryotic genomes makes them extremely useful for large scale screening of DNA, for example, in tree populations. Because it can be performed with PCR, this analysis requires only small tissue samples. Even samples that are degraded by age or environmental insult can be used. The relatively small size of microsatellite markers is advantageous for the unambiguous sizing of PCR-amplified microsatellites on polyacrylamide gels.

The screening of a microsatellite locus in different trees of a species reveals the degree of polymorphism and allelic diversity of that locus. The genetic diversity within and between populations of forestry species can thus be assessed in this manner. EST microsatellite markers may have broader uses than genomic microsatellites in assessing inter-species variability. For example, it is reported that EST microsatellite markers from sugarcane can be used for cross-species and cross-genera comparisons (Cordeiro, G. M. et al., Plant Sci. 160:1115-1123, 2001). This cross-transferability of EST microsatellite markers is important for understanding the evolution of plant species.

Microsatellite markers can also be used to identify individual trees in a breeding population insofar as they are codominant and show Mendelian inheritance.

As used herein, the term “polynucleotide” includes DNA and RNA molecules, both sense and anti-sense strands, and comprehends cDNA, genomic DNA, recombinant DNA and wholly or partially synthesized polynucleotides. A polynucleotide may consist of an entire gene, or a portion thereof. All the polynucleotides provided by the present invention are isolated and purified, as those terms are commonly used in the art.

As used herein, the term “oligonucleotide” refers to a short segment of nucleotide sequence, generally comprising between 6 and 60 nucleotides, and comprehends both probes for use in hybridization assays and primers for use in the amplification of DNA by polymerase chain reaction.

As used herein, the term “microsatellite” refers to an array of tandemly repeated nucleotide motifs, wherein each motif consists of between about 2 and about 10 basepairs. The repeats are usually uninterrupted, but may include short intervening sequences or some imperfect repeats due to, for example, point mutations, insertions or deletions.

As used herein, the term “flanking sequence” refers to the non-repetitive, nucleotide sequence adjacent to a microsatellite. “Unique flanking sequences” are those flanking sequences which are only found at one location within the genome.

As used herein, the term “polymorphic genetic marker” refers to the genetic variation seen in either microsatellites, flanking sequences or other areas in the genome DNA between different individuals or tissues. One example of a polymorphic genetic marker is the varying number of nucleotide motif repeats within a microsatellite between two plant individuals.

As used herein, the term “variant” comprehends nucleotide sequences different from the specifically identified sequences, wherein at least one nucleotide is deleted, substituted, or added. Generally, variant sequences differ from an identified sequence by substitution, deletion or addition of five nucleotides or fewer. Variants may be naturally occurring allelic variants, or non-naturally occurring variants. Preferably, variants exhibit the same functional characteristics as the inventive sequence. Variant sequences preferably exhibit at least 60%, more preferably at least 75% and, more preferably yet, at least 90% identity to a sequence of the present invention. The percentage identity is determined by aligning the two sequences to be compared, determining the number of identical residues in the aligned portion, dividing that number by the total length of the inventive, or queried, sequence and multiplying the result by 100.

Polynucleotide sequences may be aligned, and percentage of identical nucleotides in a specified region may be determined against another polynucleotide, using computer algorithms that are publicly available. Two exemplary algorithms for aligning and identifying the similarity of polynucleotide sequences are the BLASTN and FASTA algorithms. The BLASTN software is available on the NCBI anonymous FTP server under/blast/executables/. The BLASTN algorithm version 2.0.4 [Feb-24-1998], set to the default parameters described in the documentation and distributed with the algorithm, is preferred for use in the determination of variants according to the present invention. The use of the BLAST family of algorithms, including BLASTN and BLASTP, is described at NCBI's website and in the publication of Altschul, Stephen F., et al. (1997), “Gapped BLAST and PSI-BLAST: a new generation of protein database search programs”, Nucleic Acids Res. 25:3389-3402. The computer algorithm FASTA is available on the Internet. Version 2.0u4, February 1996, set to the default parameters described in the documentation and distributed with the algorithm, is preferred for the use of FASTA in the determination of variants according to the present invention. The use of the FASTA algorithm is described in W. R. Pearson and D. J. Lipman, “Improved Tools for Biological Sequence Analysis,” Proc. Natl. Acad. Sci. USA 85:2444-2448 (1988) and W. R. Pearson, “Rapid and Sensitive Sequence Comparison with FASTP and FASTA,” Methods in Enzymology 183:63-98 (1990).

The following running parameters are preferred for determination of alignments and similarities using BLASTN that contribute to the E values and percentage identity: Unix running command: blastall -p blastn -d embldb -e 10 -G 1 -E 1 -r 2 -v 50 -b 50 -i queryseq -o results; and parameter default values:

-p Program Name [String]

-d Database [String]

-e Expectation value (E) [Real]

-G Cost to open a gap (zero invokes default behavior) [Integer]

-E Cost to extend a gap (zero invokes default behavior) [Integer]

-r Reward for a nucleotide match (blastn only) [Integer]

-v Number of one-line descriptions (V) [Integer]

-b Number of alignments to show (B) [Integer]

-i Query File [File In]

-o BLAST report Output File [File Out] Optional

The BLASTN and FASTA algorithms also produce “Expect” values for alignments. The Expect value (E) indicates the number of hits one can “expect” to see over a certain number of contiguous sequences by chance when searching a database of a certain size. The Expect value is used as a significance threshold for determining whether the hit to a database, such as the preferred EMBL database, indicates true similarity. For example, an E value of 0.1 assigned to a hit is interpreted as meaning that, in a database of the size of the EMBL database, one might expect to see 0.1 matches over the aligned portion of the sequence with a similar score simply by chance. By this criterion, the aligned and matched portions of the sequences then have a probability of 90% of being the same. For sequences having an E value of 0.01 or less over aligned and matched portions, the probability of finding a match by chance in the EMBL database is 1% or less using the BLASTN or FASTA algorithm.

According to one embodiment, “variant” polynucleotides, with reference to each of the polynucleotides of the present invention, preferably comprise sequences having the same number or fewer nucleic acids than each of the polynucleotides of the present invention and producing an E value of 0.01 or less when compared to the polynucleotide of the present invention. That is, a variant polynucleotide is any sequence that has at least a 99% probability of being the same as the polynucleotide of the present invention, measured as having an E value of 0.01 or less using the BLASTN or FASTA algorithms set at the default parameters. According to a preferred embodiment, a variant polynucleotide is a sequence having the same number or fewer nucleic acids than a polynucleotide of the present invention that has at least a 99% probability of being the same as the polynucleotide of the present invention, measured as having an E value of 0.01 or less using the BLASTN or FASTA algorithms set at the default parameters.

Variant polynucleotide sequences will generally hybridize to the recited polynucleotide sequence under stringent conditions. As used herein, “stringent conditions” refers to prewashing in a solution of 6× SSC, 0.2% SDS; hybridizing at 65° C., 6× SSC, 0.2% SDS overnight; followed by two washes of 30 minutes each in 1× SSC, 0.1% SDS at 65° C. and two washes of 30 minutes each in 0.2× SSC, 0.1% SDS at 65° C.

While the DNA sequences provided by the present invention were isolated from Pinus radiata and Eucalyptus grandis, variants of the isolated sequences from other eucalyptus and pine species, as well as from other commercially important plant species, are contemplated. These include, but are not limited to, the following gymnosperms: loblolly pine Pinus taeda, slash pine Pinus elliotti, sand pine Pinus clausa, longleaf pine Pinus palustrus, shortleaf pine Pinus echinata, ponderosa pine Pinus ponderosa, Jeffrey pine Pinus jeffrey, red pine Pinus resinosa, pitch pine Pinus rigida, jack pine Pinus banksiana, pond pine Pinus serotina, Eastern white pine Pinus strobus, Western white pine Pinus monticola, sugar pine Pinus lambertiana, Virginia pine Pinus virginiana, lodgepole pine Pinus contorta, Caribbean pine Pinus caribaea, P. pinaster, Calabrian pine P. brutia, Afghan pine P. eldarica, Coulter pine P. coulteri, European pine P. nigra and P. sylvestris; Douglas-fir Pseudotsuga menziesii; the hemlocks which include Western hemlock Tsuga heterophylla, Eastern hemlock Tsuga canadensis, Mountain hemlock Tsuga mertensiana; the spruces which include the Norway spruce Picea abies, red spruce Picea rubens, white spruce Picea glauca, black spruce Picea mariana, Sitka spruce Picea sitchensis, Englemann spruce Picea engelmanni, and blue spruce Picea pungens; redwood Sequoia sempervirens; the true firs include the Alpine fir Abies lasiocarpa, silver fir Abies amabilis, grand fir Abies grandis, nobel fir Abies procera, white fir Abies concolor, California red fir Abies magnifica, and balsam fir Abies balsamea, the cedars which include the Western red cedar Thuja plicata, incense cedar Libocedrus decurrens, Northern white cedar Thuja occidentalis, Port Orford cedar Chamaecyparis lawsoniona, Atlantic white cedar Chamaecyparis thyoides, Alaska yellow-cedar Chamaecyparis nootkatensis, and Eastern red cedar Huniperus virginiana; the larches which include Eastern larch Larix laricina, Western larch Larix occidentalis, European larch Larix decidua, Japanese larch Larix leptolepis, and Siberian larch Larix sibirica; bold cypress Taxodium distichum and Giant sequoia Sequoia gigantea; and the following angiosperms, by way of example:

Eucalyptus alba, E. bancroftii, E. botyroides, E. bridgesiana, E. calophylla, E. camaldulensis, E. citriodora, E. cladocalyx, E. coccifera, E. curtisii, E. dalrympleana, E. deglupta, E. delagatensis, E. diversicolor, E. dunnii, E. ficifolia, E. globulus, E. gomphocephala, E. gunnii, E. henryi, E. laevopinea, E. macarthurii, E. macrorhyncha, E. maculata, E. marginata, E. megacarpa, E. melliodora, E. nicholii, E. nitens, E. nova-angelica, E. obliqua, E. obtusiflora, E. oreades, E. pauciflora, E. polybractea, E. regnans, E. resinifera, E. robusta, E. rudis, E. saligna, E. sideroxylon, E. stuartiana, E. tereticornis, E. torelliana, E. urnigera, E. urophylla, E. viminalis, E. viridis, E. wandoo and E. youmanni.

The inventive sequences may be isolated by high throughput sequencing of cDNA libraries from the target species, for example Eucalyptus grandis and Pinus radiata, as described below in Examples 1 and 2. Alternatively, oligonucleotide probes based on the sequences provided in SEQ ID NO: 1-1054 can be synthesized and used to identify positive clones in either cDNA or genomic DNA libraries from target species, such as Eucalyptus grandis and Pinus radiata, by means of hybridization techniques. Alternatively, PCR may be employed to specifically amplify polynucleotides of the present invention, using oligonucleotide primers designed to the inventive sequences. Oligonucleotide probes and/or primers can be shorter than the sequences provided herein but should be at least about 6 nucleotides, preferably at least about 10 nucleotides and most preferably at least about 20 nucleotides in length. Hybridization and PCR techniques suitable for use with such oligonucleotide probes and primers are well known in the art. Positive clones may be analyzed by restriction enzyme digestion, DNA sequencing or other methods well known in the art.

In addition, the DNA sequences of the present invention may be generated by synthetic means using techniques well known in the art. Equipment for automated synthesis of oligonucleotides is commercially available from suppliers such as Perkin Elmer/Applied Biosystems Division (Foster City, Calif.) and may be operated according to the manufacturer's instructions.

DNA constructs comprising the inventive polynucleotides are also provided, together with host cells transformed with such constructs. Such DNA constructs generally include at least one sequence of the present invention combined with, or contiguous with, other sequences which may or may not be related to the inventive sequence. DNA constructs comprising the disclosed polynucleotides may be employed, for example, to introduce microsatellite markers into transgenic plants for use as polymorphic identification tags in promoter areas, with different transgenic plants containing microsatellites of varying size but identical flanking sequences. Techniques for preparing such DNA constructs and for transforming plants using such constructs are well known in the art and include, for example, those described in Gleave, A. P. 1992, Plant Mol. Biol. 20:1203-1207; and Janssen, B.-J. and Gardner, R. C. 1989, Plant Mol. Biol. 14:61-72.

The polynucleotide sequences of the present invention may be employed to design oligonucleotide for use as primers and/or probes in polymorphism detection using standard techniques, such as polymerase chain reaction (PCR), or DNA-DNA, DNA-RNA or RNA-RNA hybridization. The oligonucleotide probes and/or primers, which generally comprise between about 6 and about 60 nucleotides, may contain part or all of a microsatellite repeat contained within the inventive polynucleotide sequence, or a sequence complementary thereto, in addition to at least a portion of the corresponding flanking sequence. However, for PCR amplification, the oligonucleotide primer sequence is preferably at least about 10 nucleotides distant from the repeat into the flanking sequence.

In a preferred embodiment, oligonucleotide primers and/or probes for use in the inventive methods comprise at least about 6 contiguous nucleotides, more preferably at least about 10 contiguous nucleotides and most preferably at least about 20 contiguous nucleotides of sequence complementary to a polynucleotide sequence provided herein. The sensitivity and specificity of the oligonucleotide primer/probe are determined by the primer/probe length and the uniqueness of a sequence within a given sample of DNA. The oligonucleotide primer or hybridization probe may occur naturally and may be isolated, for example, from a restriction digest, or may be produced synthetically using methods well known in the art.

The term “oligonucleotide primer” as used herein refers to a polynucleotide which is capable of acting as an initiation point for synthesis of either DNA or RNA when placed under conditions which induce synthesis of a primer extension product complementary to a specific nucleic acid strand. As used herein, the term “extension product” refers to the nucleotide sequence which is synthesized from the 3′ end of the oligonucleotide primer and which is complementary to the strand to which the oligonucleotide primer is bound. The exact length of an oligonucleotide primer will depend on many factors relating to the ultimate function and use of the primer. In a preferred embodiment, the oligonucleotide primer is a single-stranded polynucleotide of sufficient length to prime the synthesis of an extension product from a specific sequence in the presence of an inducing agent. As noted above, the oligonucleotide primers of the present invention are at least about 6 nucleotides in length.

An oligonucleotide primer pair is selected to detect a specific microsatellite. Each primer of each pair is selected to be complementary to a different strand in the flanking sequence or a variant of a flanking sequence of each specific microsatellite sequence to be amplified. Thus, one primer of each pair is sufficiently complementary to hybridize with a part of the sequence in the sense strand and the other primer is sufficiently complementary to hybridize with a different part of the same sequence in the antisense strand. Although the primer sequence need not reflect the exact sequence of the naturally occurring flanking sequence, the more closely the 3′ end reflects the exact sequence, the better the binding during the annealing stage. Differential labels may be employed, as described for example in U.S. Pat. No. 5,364,759, to distinguish extension products from each other.

Techniques for PCR based assays are well known in the art (see, for example, Mullis, et al., Cold Spring Harbor Symp. Quant. Biol., 51:263, 1987; Erlich ed., PCR Technology, Stockton Press, NY, 1989). Following DNA amplification by PCR using oligonucleotide primers specific for a given microsatellite, the amplified DNA is separated according to size by, for example, gel electrophoresis. The separated DNA may then be examined for DNA length polymorphism. Restriction digestion and sequencing of PCR products, using techniques well known in the art, may be used to obtain more information for fingerprinting and mapping purposes. The inventive methods may thus be used for genetic analysis of DNA from a single plant, or for the detection and quantification of target DNA within pooled DNA from several plants. For a review of the use of microsatellite sequences and associated flanking sequences in PCR techniques see Weising K, Atkinson R G, Gardner R C, 1995, Genomic fingerprinting by microsatellite-primed PCR: a critical evaluation. PCR Methods Appl. 4(5): 249-255.

The oligonucleotide primers of the present invention may also be employed to detect the presence of DNA from a specific plant from a sample of DNA using PCR. The feasibility of this kind of assay has been demonstrated by Groppe et al. (1997 Appl. Environ. Microbiol. 63(4): 1543-1550), who amplified as little as 1.0 pg of a specific fungal DNA from a mixture of 100 ng of DNA of plant origin using microsatellite-primed PCR.

Oligonucleotide probes containing at least a portion of a polynucleotide sequence of the present invention may be employed to probe restriction digests of plant DNA using nucleic acid hybridization techniques well known in the art, such as Southern, Northern and in situ hybridizations (Maniatis et al., Molecular Cloning—A Laboratory Manual, Cold Spring Harbor Laboratory, Cold Spring Harbor, N.Y., 1989). In this manner, the inventive sequences may be employed as hybridization probes for oligonucleotide fingerprinting as described, for example, by Weising et al. (Weising K, Beyermann B, Ramsser J & Kahl G 1991 Electrophoresis 12: 159-169), or for library screening, as described, for example, by Wu & Tanksley (1993 Mol Gen. Genet. 241: 225-235).

The DNA sample to be tested using the methods described herein is preferably plant genomic DNA, but may also be a cDNA or other representative DNA sample. Preferably, the DNA is from a plant of the genus Eucalyptus or Pinus, and more preferably from a plant of the species Eucalyptus grandis or Pinus radiata. The DNA may be isolated from any part of the plant, including the fruit or seeds, using methods well known in the art.

The word “about,” when used in this application with reference to a number of nucleotide residues, contemplates a variance of up to 3 residues from the stated number. The word “about” when used with reference to a percentage identity of nucleotides, contemplates a variance of up to 3% from the stated percentage.

The following examples are offered by way of illustration and not by way of limitation.

### EXAMPLE 1

### Isolation and Characterization of cDNA Sequences from Eucalyptus grandis and Pinus radiata

Eucalyptus grandis cDNA expression libraries were constructed and screened as follows.

mRNA was extracted from the plant tissue using the protocol of Chang et al. (Plant Molecular Biology Reporter 11:113-116 (1993)) with minor modifications. Specifically, samples were dissolved in CPC-RNAXB (100 mM Tris-Cl, pH 8,0; 25 mM EDTA; 2.0 M NaCl; 2% CTAB; 2% PVP and 0.05% Spermidine*3 HCl) and extracted with chloroform:isoamyl alcohol, 24:1. mRNA was precipitated with ethanol and the total RNA preparate was purified using a Poly(A) Quik mRNA Isolation Kit (Stratagene, La Jolla, Calif.). A cDNA expression library was constructed from the purified mRNA by reverse transcriptase synthesis followed by insertion of the resulting cDNA clones in Lambda ZAP using a ZAP Express cDNA Synthesis Kit (Stratagene), according to the manufacturer's protocol. The resulting cDNAs were packaged using a Gigapack II Packaging Extract (Stratagene) employing 1 μl of sample DNA from the 5 μl ligation mix. Mass excision of the library was done using XL1-Blue MRF′ cells and XLOLR cells (Stratagene) with ExAssist helper phage (Stratagene). The excised phagemids were diluted with NZY broth (Gibco BRL, Gaithersburg, Md.) and plated out onto LB-kanamycin agar plates containing X-gal and isopropylthio-beta-galactoside (IPTG).

Of the colonies plated and picked for DNA miniprep, 99% contained an insert suitable for sequencing. Positive colonies were cultured in NZY broth with kanamycin and cDNA was purified by means of alkaline lysis and polyethylene glycol (PEG) precipitation. Agarose gel at 1% was used to screen sequencing templates for chromosomal contamination. Dye primer sequences were prepared using a Turbo Catalyst 800 machine (Perkin Elmer/Applied Biosystems, Foster City, Calif.) according to the manufacturer's protocol.

DNA sequence for positive clones was obtained using a Perkin Elmer/Applied Biosystems Division Prism 377 sequencer. cDNA clones were sequenced from the 5′ end.

The resulting cDNA sequences were searched for the presence of short tandem repeats, or microsatellites, by computer analysis. The DNA sequence of each microsatellite isolated from Eucalyptus grandis and its flanking sequence(s) are provided in SEQ ID NO: 1-24 and 26-1006. Each of these sequences was compared to known sequences in the EMBL DNA database (vs. 52+updates to January 1998) using the BLASTN algorithm. Multiple alignments of redundant sequences were used to detect additional microsatellite-containing sequences.

Pinus radiata cDNA expression libraries were constructed from various tissues and screened as described above. DNA sequences for positive clones was obtained using forward and reverse primers on an Applied Biosystems Prism 377 sequencer and the determined sequences were compared to known sequences in the database as described above. The DNA sequences of each microsatellite containing sequence isolated from Pinus radiata are provided in SEQ ID NO: 25 and 1007-1054.

### EXAMPLE 2

### PCR Amplification and Polymorphism Analysis of Pinus radiata DNA for Detecting Genetic Variation Between Germplasms of Different Origins

The inventive DNA sequences may be used to detect genetic variation between germplasms of different origins as follows.

PCR primers are designed from the flanking sequences provided in SEQ ID NO: 1-1054, so that the amplification product is a few hundred basepairs or less. Primer selection is made from the inventive sequences by using PCR primer determination software generally available and well known in the art, such as AMPLIFY software (Hillier L & Green P. 1991. OSP: A computer program for choosing PCR and DNA sequencing primers. PCR Methods and Applications 1:124-128). The designed primers are synthesized using, for example, equipment available from Perkin Elmer/Applied Biosystems Division, according to the manufacturer's protocol. Genomic DNA samples are isolated from different Pinus radiata individuals and amplified using standard PCR protocols with the designed primers.

The amplified DNA product is electrophoresed using standard protocols for separation of the variously sized polymorphic DNAs of different germplasm samples. The polymorphic bands are visualized by means of UV light with ethidium bromide staining or by other standard DNA staining/detection methods. The bands are then scored either visually or by computer-aided image analysis and the data obtained across pine tree individuals are compared.

Although the present invention has been described in some detail by way of illustration and example for purposes of clarity of understanding, changes and modifications can be carried out without departing from the scope of the invention which is intended to be limited only by the scope of the appended claims. For example, other possible ways of using the microsatellite-containing sequences provided by the present invention will be readily apparent to others of skill in the art, including plant breeders doing marker assisted selection.

We claim:

1. An isolated polynucleotide comprising a sequence selected from the group consisting of:
(a) sequences provided in SEQ ID NO: 1-1054; and
(b) sequences complementary to a sequence provided in SEQ ID NO: 1-1054;
(c) sequences having at least about a 99% probability of being the same as a sequence of (a) or (b) as measured using computer algorithm BLASTN;
(d) sequences having at least 75% identity to a sequence of (a) or (b); and
(e) sequences having at least 90% identity to a sequence of (a) or (b).

2. An isolated polynucleotide comprising a sequence selected from the group consisting of:
(a) left flanking sequences of DNA sequences provided in SEQ ID NO: 1-1054;
(b) sequences complementary to a sequence of (a); and
(c) sequences having at least a 99% probability of being the same as a sequence of (a) or (b);
(d) sequences having at least 75% identity to a sequence of (a) or (b); and
(e) sequences having at least 90% identity to a sequence of (a) or (b).

3. An isolated polynucleotide comprising a sequence selected from the group consisting of:
(a) right flanking sequences of DNA sequences provided in SEQ ID NO: 1-1054;
(b) sequences complementary to a sequence of (a); and
(c) sequences having at least a 99% probability of being the same as a sequence of (a) or (b);
(d) sequences having at least 75% identity to a sequence of (a) or (b); and
(e) sequences having at least 90% identity to a sequence of (a) or (b).

4. An isolated polynucleotide comprising a sequence selected from the group consisting of:
(a) at least three contiguous repeats of a sequence provided in SEQ ID NO: 1055;
(b) at least three contiguous repeats of a sequence provided in SEQ ID NO: 1056;
(c) at least three contiguous repeats of a sequence provided in SEQ ID NO: 1057;
(d) sequences complementary to a sequence of (a), (b) or (c); and
(e) sequences having at least a 99% probability of being the same as a sequence of (a), (b) or (c);
(f) sequences having at least 75% identity to a sequence of (a), (b) or (c); and
(g) sequences having at least 90% identity to a sequence of (a), (b) or (c).

5. An oligonucleotide primer that binds specifically to an isolated polynucleotide selected from the group consisting of SEQ ID NO: 1-1054, wherein the oligonucleotide primer comprises at least 6 contiguous nucleotides of a sequence complementary to a sequence provided in SEQ ID NO: 1-1054.

6. An oligonucleotide primer that binds specifically to an isolated polynucleotide selected from the group consisting of SEQ ID NO: 1-1054, wherein the oligonucleotide primer comprises at least 10 contiguous nucleotides of a sequence complementary to a sequence provided in SEQ ID NO: 1-1054.

7. An oligonucleotide primer that binds specifically to an isolated polynucleotide selected from the group consisting of SEQ ID NO: 1-1054, wherein the oligonucleotide primer comprises at least 20 contiguous nucleotides of a sequence complementary to a sequence provided in SEQ ID NO: 1-1054.

8. An isolated oligonucleotide primer pair selected from the group consisting of:
(a) at least 10 contiguous nucleotides of a left flanking sequence provided in Table 1 and at least 10 contiguous nucleotides of a right flanking sequence provided in Table 2, wherein the left flanking sequence and right flanking sequence are associated with the same SEQ ID NO:;
(b) a sequence pair complementary to a sequence pair of (a); and
(c) a sequence pair having at least 90% identity to a sequence pair of (a) or (b).

9. A method for detecting a polymorphic genetic marker in a subject, comprising:
(a) isolating DNA from the subject;
(b) contacting the isolated DNA with an oligonucleotide primer pair according to claim 8 in a polymerase chain reaction to provide amplified DNA molecules;
(c) separating the amplified DNA molecules according to size; and
(d) analyzing the amplified DNA molecules for the presence of the polymorphic genetic marker.

10. A method for detecting a polymorphic genetic marker in a subject, comprising:
(a) isolating DNA from the subject; and
(b) analyzing the isolated DNA for the presence of the polymorphic genetic marker using at least one oligonucleotide to detect the polymorphic marker, wherein the oligonucleotide comprises at least 6 contiguous residues of a sequence selected from the group consisting of: (i) sequences provided in SEQ ID NO: 1-1054; (ii) sequences complementary to a sequence of SEQ ID NO: 1-1054; and (iii) sequences having at least 90% identity to a sequence of (i) or (ii).

11. The method of claim 10, wherein the oligonucleotide comprises at least about 20 contiguous nucleotides of a sequence selected from the group consisting of:
(i) sequences provided in SEQ ID NO: 1-1054;
(ii) sequences complementary to a sequence of SEQ ID NO: 1-1054; and
(iii) sequences having at least 90% identity to a sequence of (i) or (ii).

12. The method of any one of claims 10 and 11 wherein the subject is selected from the group consisting of plants, fruit and seeds.

13. The method of claim 12, wherein the subject is a woody plant.

14. The method of claim 12, wherein the plant is selected from the group consisting of eucalyptus and pine.

15. The method of claim 10, wherein step (b) further comprises:
(a) amplifying DNA molecules from the isolated DNA by polymerase chain reaction using the oligonucleotide as a primer;
(b) separating the amplified DNA molecules according to size; and
(c) analyzing the amplified DNA molecules for the presence of the polymorphic genetic marker.

16. The method of claim 15, wherein the amplified DNA molecules are separated by means of gel electrophoresis.

17. The method of claim 10, wherein step (b) further comprises:
(a) contacting the isolated DNA with the oligonucleotide in a hybridization assay;
(b) determining the presence of a DNA molecule that hybridizes to the oligonucleotide; and
(c) analyzing the DNA molecule for the presence of the polymorphic genetic marker.

18. A method for detecting a polymorphic genetic marker in a subject comprising:
(a) isolating DNA from the subject;
(b) contacting the isolated DNA with an oligonucleotide probe in a hybridization assay to detect the presence of DNA molecules that hybridize to the oligonucleotide probe, wherein the oligonucleotide probe is specific for a polynucleotide having a sequence selected from the group consisting of: (i) sequences provided in SEQ ID NO: 1-1054; (ii) sequences complementary to a sequence of SEQ ID NO: 1-1054; and (iii) sequences having at least 90% identity to a sequence of (i) or (ii);
(c) separating the DNA molecules according to size; and
(d) analyzing the DNA molecules for the presence of the polymorphic genetic marker.

19. The method of claim 18, wherein the oligonucleotide probe comprises at least about 6 contiguous residues of a sequence selected from the group consisting of:
(a) sequences provided in SEQ ID 1-1054, (b) sequences complementary to a sequence of (a); and
(c) sequences having at least 90% identity to a sequence of (a) or (b).

20. The method of claim 18 wherein the subject is selected from the group consisting of plants, fruit and seeds.

21. The method of claim 20, wherein the subject is a woody plant.

22. The method of claim 21, wherein the plant is selected from the group consisting of eucalyptus and pine.

23. The method of claim 18, wherein the amplified DNA molecules are separated by means of gel electrophoresis.

24. A kit for detecting a polymorphic genetic marker comprising a container which holds at least one isolated polynucleotide according to any one of claims 1-4.

25. A kit for detecting a polymorphic genetic marker comprising a container which holds at least one oligonucleotide primer according to any one of claims 5-7.
